(19) United States
(12) Patent Application Publication
(10) Pub. No.: US 2005/0014706 A1
(43) Pub. Date: Jan. 20, 2005

(54) Method of treating tuberculosis

(76) Inventors: Kanakeshwari Falzari, Forest Park, IL (US); Scott G. Franzblau, Chicago, IL (US); Zhaohai Zhu, Grayslake, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/889,346

(22) Filed: Jul. 12, 2004

Related U.S. Application Data
(60) Provisional application No. 60/486,979, filed on Jul. 14, 2003.

(51) Int. Cl. A61K 31/7048; A61K 31/365

(52) U.S. Cl. 514/28; 514/450

(57) ABSTRACT

Macrolide and ketolides, and compositions containing the same, useful in the treatment of tuberculosis are disclosed. Methods of treating tuberculosis using the macrolides and ketolides, and compositions containing the same, also are disclosed.

### CROSS REFERENCE TO RELATED APPLICATION

This application claims the benefit of U.S. provisional patent application Ser. No. 60/486,979, filed Jul. 14, 2003.

### FIELD OF THE INVENTION

The present invention relates to methods of treating tuberculosis. More particularly, the present invention relates to a method of treating tuberculosis comprising administrating a therapeutically effective amount of a macrolide, a ketolide, or mixtures thereof, or a composition containing a macrolide, a ketolide, or mixtures thereof, to an individual in need thereof.

### BACKGROUND OF THE INVENTION

Tuberculosis (TB) is an infectious disease that usually attacks the lungs, but is capable of attacking most parts of the body. Tuberculosis is spread from person to person through the air. When individuals infected with TB cough, laugh, sneeze, sing, or talk, TB bacteria can be spread into the air. If a second person inhales TB bacteria, a possibility exists that the second person also will become infected with tuberculosis. However, repeated contact typically is required for infection.

Medical experts estimate that about 10 million Americans are infected with TB bacteria, and about 10 percent of these people will develop active TB in their lifetime. However, TB is an increasing worldwide problem, especially in Africa. It is estimated that, worldwide, about one billion people will become newly infected, over 150 million people will contract active TB, and 36 million people will die between now and 2020 unless TB control is improved.

An individual infected with TB, but not suffering from TB disease, i.e., has latent TB, can be administered preventive therapy. Preventive therapy kills bacteria in order to prevent a case of active TB. The usual treatment for latent TB is a daily dose of isoniazid (also termed “INH”).

If an individual has TB disease, i.e., has active TB, the individual typically is administered a combination of several drugs. It is very important, however, that the individual continue a correct treatment regimen for the full length of the treatment. If the drugs are taken incorrectly, or stopped, the individual can suffer a relapse and will be able to infect others with TB.

When an individual becomes sick with TB a second time, the TB infection may be more difficult to treat because the TB bacteria have become drug resistant, i.e., TB bacteria in the body are unaffected by some drugs used to treat TB. Multidrug-resistant tuberculosis (MDR TB) is a very dangerous form of tuberculosis. In particular, some TB bacteria become resistant to the effects of various anti-TB drugs, and these resistant TB bacteria then can cause TB disease. Like regular TB, MDR TB can be spread to others.

To avoid drug resistance in the treatment of TB, a four-drug regimen, i.e., isoniazid, rifampin, pyrazinamide, and streptomycin, is administered to TB patients. Aminoglycosides, such as streptomycin, are important anti-TB agents, but their utility is restricted by the requirement of parenteral administration, which is inconvenient and leads to poor patient compliance. It is theorized that poor patient compliance also can lead to the development of drug resistance, and it appears that the frequency of streptomycin resistance among anti-TB drugs is surpassed only by isoniazid.

In view of the above, an urgent need exists for new anti-TB agents useful in an effective treatment regimen for both the active and latent TB, and that effectively treat TB caused by multidrug resistant (MDR) strains of bacteria. Therefore, it would be advantageous to provide compounds and compositions for administration to an individual in the treatment of tuberculosis. As set forth in detail hereafter, the present invention is directed to the use of macrolide and ketolide compounds, and pharmaceutical compositions containing the same, useful in methods of treating tuberculosis.

### SUMMARY OF THE INVENTION

The present invention is directed to a method of treating tuberculosis (TB). More particularly, the present invention is directed to a method of treating latent, active, and multidrug-resistant TB by administering a therapeutically effective amount of a macrolide, a ketolide, or mixtures thereof, to a mammal in need thereof.

Accordingly, one aspect of the present invention is to provide a method of treating TB in a mammal, including humans.

Another aspect of the present invention is to provide a pharmaceutical composition comprising a macrolide, ketolide, or mixtures thereof that can be administered to an individual in a therapeutically effective amount to treat latent, active, or multi-drug-resistant TB. In preferred embodiments, the macrolide or ketolide has an MIC vs. M. tuberculosis of about 50 μM or less, e.g., about 0.01 nM to about 50 μM.

Another aspect of the present invention is to provide a method of treating TB comprising administering to a mammal in need thereof (a) a pharmaceutical composition comprising a macrolide, a ketolide, or mixtures thereof and, optionally, (b) one or more additional drugs useful in the treatment of TB.

Still another aspect of the present invention is to provide an article of manufacture comprising:
(a) a packaged pharmaceutical composition comprising a macrolide, a ketolide, or mixtures thereof;
(b) an insert providing instructions for the administration of the macrolide, ketolide, or mixture thereof to treat TB; and
(c) a container for (a) and (b).
Yet another aspect of the present invention is to provide an article of manufacture comprising
(a) a packaged composition comprising a macrolide, a ketolide, or mixtures thereof;
(b) a packaged composition comprising a second therapeutic agent useful in a treatment of tuberculosis;
(c) an insert providing instructions for a simultaneous or sequential administration of (a) and (b) to treat tuberculosis; and
(d) a container for (a), (b), and (c).

These and other aspects and advantages of the present invention will become apparent from the following detailed description of the preferred embodiments.

### DETAILED DESCRIPTION OF THE PREFERRED EMBODIMENTS

The macrolide classes of clinically useful antimicrobial agents fail to include tuberculosis as a treatable indication. Consequently, macrolides and ketolides, including derivatives of known macrolides and ketolides, were investigated for the possibility of providing a clinically useful anti-TB drug.

Erythromycin and related macrolide antibiotics are among the safest and most effective treatments for diseases caused by Streptococci and Staphylococci bacteria. Although some of these antibiotics are active against some related mycobacteria, such as those that cause leprosy, other skin infections, and opportunistic infections in HIV/AIDS, these antibiotic are not clinically useful for the treatment of tuberculosis. The newest macrolides, designed to overcome resistance of Staph and Strep, are not themselves active against M. tuberculosis, but it now has been found that related compounds possess potent anti-TB activity.

Although previous macrolides have not shown potent anti-TB activity, the macrolides as a class are attractive compounds for treating TB because of the following properties: (a) excellent oral bioavailability and distribution to the lungs, (b) low toxicity, (c) infrequent adverse reactions, (d) extensive intracellular concentration and activity, and (e) a demonstrated clinical utility and bactericidal activity in infections caused by M. avium and M. leprae. In addition, erythromycin is a relatively inexpensive starting material for the preparation of new anti-TB macrolides, and a majority of the synthetic routes are relatively short, e.g., typically 10 steps or less. Thus, a new anti-TB drug arising from this class of compounds is considered to be economically viable.

Erythromycin, a prototypical first generation macrolide, is a natural product derived from Streptomyces erythreus. Erythromycin interferes with protein synthesis on the 50S subunit of 70S ribosomes by binding in the peptidyl transferase center and blocking the movement of proteins through the exit tunnel. Erythromycin originally was used as an alternative agent in the treatment of patients with infections caused by Staphylococcus and Streptococcus species, but who were allergic to β-lactams. Erythromcyin possesses most of the favorable macrolide properties mentioned above, but suffers from a short serum half-life, thus necessitating tid or qid dosing (i.e., three or four times per day, respectively), and acid lability, the product of which leads to gastric motility-based discomfort. In addition, erythromycin activity is restricted to controlling Gram positive bacteria.

Second generation macrolides were developed to have superior acid stability and serum half-life. Clarithromycin, roxithromycin, and azithromycin are examples of such second generation macrolides. 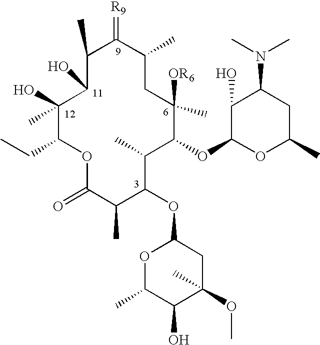
R₆, R₉=H, O: Erythromycin
R₆, R₉=Me, O: Clarithromycin
R₆, R₉=H, NOCH₂O (CH₂)₂OMe: Roxithromycin

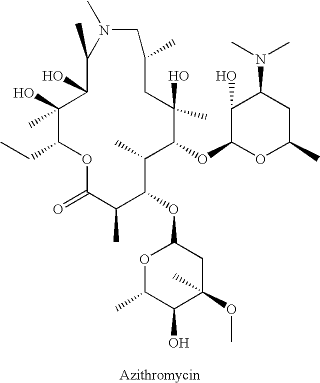

When the second generation macrolides entered phase II and III trials for infections caused by Gram positive and Gram negative pathogens, M. avium was becoming recognized as a significant pathogen in HIV-infected individuals, and in vitro metabolic assays were being developed for the unculturable M. leprae, which facilitated the search for new bactericidal drugs to shorten the treatment duration. It became apparent that the second generation macrolides, i.e., clarithromycin and azithromycin, along with rifabutin, were the most active clinical agents against M. avium (G. W. Amsden et al., Drugs, 54:69-80 (1997)).

With the exception of azithromycin, these compounds also were found to possess potent activity against M. leprae in axenic media (S. G. Franzblau, Antimicrob Agents Chemother, 33:2115-7 (1989); S. G. Franzblau et al., Antimicrob Agents Chemother, 32:1758-62 (1988)), in macrophages (N. Ramasesh et al., Antimicrob Agents Chemother, 33:657-62 (1989)), mice (S. G. Franzblau et al., Antimicrob Agents Chemother, 32:1758-62 (1988); B. Ji et al., Antimicrob Agents Chemother, 40:393-9 (1996)), and ultimately in clinical trials (G. P. Chan et al., Antimicrob Agents Chemother, 38:515-7 (1994); B. Ji et al., Antimicrob Agents Chemother, 40:2137-41 (1996); T. H. Rea, Int J Lepr Other Mycobact Dis, 68:129-35 (2000)). Clarithromycin currently is recommended by the World Health Organization for treatment of leprosy in cases of rifampin resistance or intolerance (J. H. Grosset, Int J Lepr Other Mycobact Dis, 69:S14-8 (2001)). Other studies demonstrated low MICs (minimum inhibitory concentrations) and/or a clinical utility of second generation macrolides against M. kansasii (R. S. Witzig et al., Antimicrob Agents Chemother, 37:1997-9 (1993)), M. marinum (A. Aubry et al., Arch Intern Med, 162:1746-52 (2002); A. Aubry et al., Antimicrob Agents Chemother, 44:3133-6 (2000); M. Braback et al., Antimicrob Agents Chemother, 46:1114-6 (2002); B. A. Brown et al., Antimicrob Agents Chemother, 36:1987-90 (1992)), and other mycobacterial opportunistic pathogens (B. A. Brown et al., Antimicrob Agents Chemother, 36:1987-90 (1992); N. Rastogi et al., Antimicrob Agents Chemother, 36:2841-2 (1992)).

The impressive activity of second generation macrolides did not include an activity against M. tuberculosis. The MIC of clarithromycin against the tubercle bacillus ranges from 4-64 μg/ml, and activity in mouse models was marginal (J. Luna-Herrera et al., Antimicrob Agents Chemother, 39:2692-5 (1995)) to nil (C. Truffot-Pernot et al., Antimicrob Agents Chemother, 39:2827-8 (1995)). Indeed the marginal activity reported by Luna-Herrera et al. (1995) is attributed to excellent distribution to the lungs and intracellular concentration. This study, together with a demonstrated activity in leprosy and M. avium infection, formed the basis for its anecdotal use in treating MDR-TB when therapeutic options are extremely limited (C. Mitnick et al., N Engl J Med, 348:119-28 (2003)). Clearly, in vitro and in vivo results indicate that clarithromycin cannot be expected to offer significant clinical benefits in the treatment of tuberculosis.

The innate resistance of M. tuberculosis to clarithromycin apparently is attributed to the same resistance factors encountered in Staph and Strep, e.g., methylation of A2058 by a ribosome methylase (Rv1988) and possibly efflux (Rv0037c). The existence of an erythromycin esterase (Rv2030c) also could be a factor. M. bovis BCG and M. leprae are highly susceptible to clarithromycin, and each lack and each homologous genes.

The third generation of macrolides (J. M. Blondeau, Expert Opin Pharmacother, 3:1131-51 (2002); J. M. Blondeau et al., Expert Opin Investig Drugs, 11:189-215 (2002); G. G. Zhanel et al., Expert Opin Pharmacother, 3:277-97 (2002); G. G. Zhanel et al., Drugs, 62:1771-804 (2002)) are represented largely by the ketolides, and were developed with the intention of overcoming the ribosome modification and efflux resistance mechanisms found in Gram positive cocci. The 3-cladinose was hydrolyzed and the resulting 3-hydroxyl group was oxidized to a 3-carbonyl group. Structures of representative ketolides are:

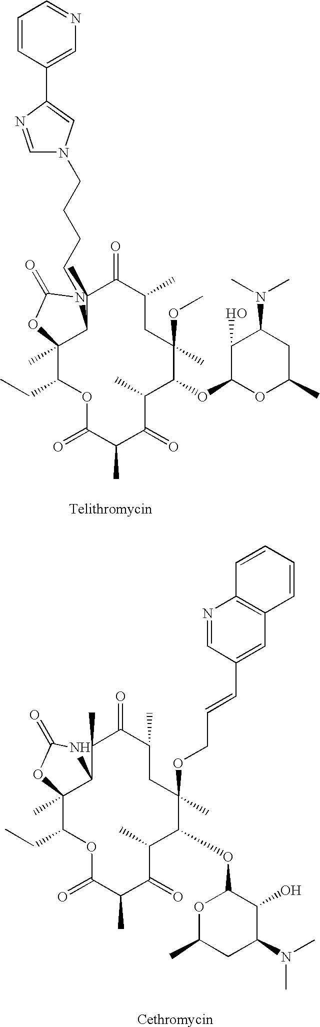

Telithromycin was the first commercial third generation macrolide. Removal of the cladinose precludes active efflux, while the 11,12-carbamate substitution both precludes inducible ribosome modification and increases binding affinity in the peptidyl transfer site of the ribosome in the case of constitutively methylated ribosomes. The ketolide, cethromycin (ABT-773), used a 6-position substitution to overcome ribosome modification.

A comparative study of the antimycobacterial activity of clarithromycin vs. telithromycin (as well as the fluorinated analog of telithromycin, HMR or RU 3004) revealed a superior activity of clarithromycin for the moderately clarithromycin-susceptible mycobacteria M. bovis BCG, M. avium, M. ulcerans, and M. paratuberculosis, the clarithro-mycin-resistant mycobacteria M. tuberculosis, M. bovis, M. africanum, and M. simiae (N. Rastogi et al., Antimicrob Agents Chemother, 44:2848-52 (2000)).

The present invention is directed to a method of treating tuberculosis utilizing a macrolide, a ketolide, or mixtures thereof. The macrolide, ketolide, or mixtures thereof can be used neat, or incorporated into a pharmaceutical preparation. The present method can utilize a single macrolide, a mixture of macrolides, a single ketolide, a mixture of ketolides, or a mixture of a macrolide and a ketolide.

Useful macrolides are disclosed herein and, for example, in U.S. Pat. Nos. 5,439,889; 5,786,339; 5,543,400; and 6,096,714; and in WO 02/32919, each incorporated herein by reference. More particularly, in one embodiment, the macrolide comprises a compound disclosed in U.S. Pat. No. 5,543,400, having a structural formula: 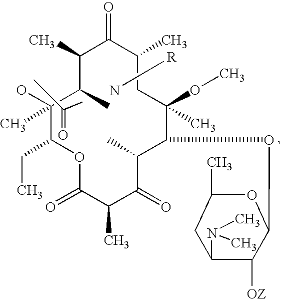
wherein R is 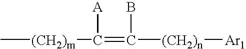
   m and n are individually integers from 0 to 6, A and B are individually a member selected from the group consisting of hydrogen, halogen, and alkyl of 1 to 8 carbon atoms, the double bond geometry being E or Z or E+Z or A and B for a third bond between the carbon atoms to which they are attached, Ar is selected from the group consisting of a) carbocyclic aryl or up to 18 carbon atoms optionally substituted with at least one member of the group consisting of free carboxy, alkoxycarbonyl, carboxy salified with a nontoxic, pharmaceutically acceptable base, amidified carboxy, —OH, halogen, —NO₂, —CN, alkyl, alkenyl, alkynyl, alkoxy, alkenyloxy, alkynyloxy, alkylthio, alkenylthio, and alkynylthio of up to 12 carbon atoms, N-alkyl, N-alkenyl, and N-alkynyl of up to 12 carbon atoms and cycloalkyl of 3 to 12 carbon atoms, all optionally substituted with at least one halogen and 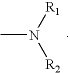
   R₁ and R₂ are individually selected from the group consisting of hydrogen, alkyl of 1 to 12 carbon atoms, carbocyclic aryl, aryloxy, arylthio, heterocyclic aryl, and aryloxy and arylthio containing at least one heteroatom, all optionally substituted as above and b) heterocyclic aryl having at least one heteroatom optionally substituted with at least one of the above substituents, Z is hydrogen or acyl or an organic carboxylic acid of 1 to 18 carbon atoms and their nontoxic, pharmaceutically acceptable acid addition salts.

In another embodiment, the macrolide comprises a compound disclosed in WO 02/32919, having a structural formula: 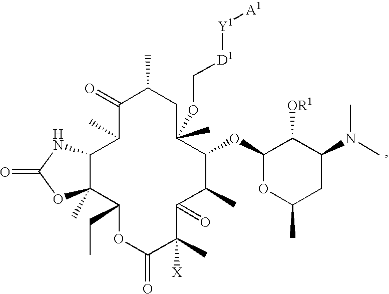
or a therapeutically acceptable salt or prodrug thereof, wherein
X is selected from hydrogen and fluoride;
D¹ is selected from CH═CH or C≡C;
Y¹ is selected from isoxazole, oxazole, isothiazole, dihydroisoxazole, and dihydrooxazole;
A¹ is selected from aryl and heteroaryl; and
R¹ is selected from hydrogen and R^{p}, wherein R^{p} is a hydroxyl protecting group.

In a third embodiment the macrolide comprises a compound disclosed in U.S. Pat. No. 5,786,339 having a structural formula: 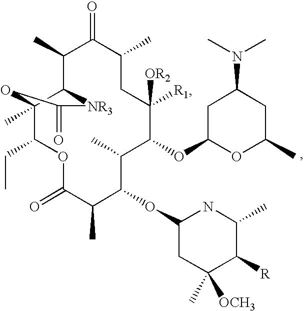
wherein R and R₁ are —OH or —O-acyl of an organic carboxylic acid of 2 to 20 carbon atoms, R₂ is hydrogen or methyl, R₃ is —(CH₂)ₘ—R₄ or 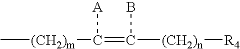
   or —N—(CH₂)_{q}—R₄, m is an integer from 1 to 6, a, p, and q are individually an integer from 0 to 6, A and B are individually selected from the group consisting of hydrogen, halogen, and alkyl of 1 to 8 carbon atoms with the geometry of the double bond being E or Z or a mixture of E and Z or A and B form a triple bond, R₄ is an optionally substituted mono- or polycyclic heterocycle and their nontoxic, pharmaceutically acceptable acid addition salts.

In a further embodiment, the macrolide comprises a compound disclosed in U.S. Pat. No. 6,096,714 having a structural formula: 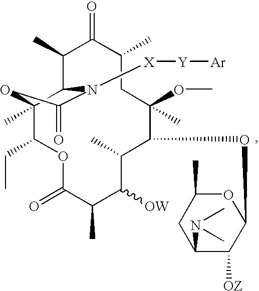
wherein X represents a CH₂ or SO₂ radical or an oxygen atom, Y represents a (CH₂)ₘ(CH═CH)ₙ(CH₂)ₒ radical, with m+n+o≦8, n=O or 1,
Ar represents an optionally substituted aryl radical,
W represents a hydrogen atom or the remainder of a carbamate function 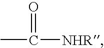
   wherein R″ represents an alkyl radical containing up to 8 carbon atoms or an optionally substituted aryl radical, and
Z represents a hydrogen atom or the remainder of an acid, as well as their addition salts with acids.

In a fifth embodiment, the macrolide comprises a compound disclosed in U.S. Pat. No. 5,439,889 having a structural formula: 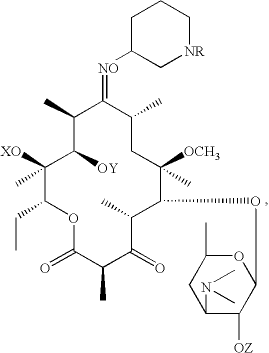
wherein X and Y are hydrogen or together form 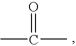
   R is 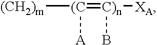
   m is an integer from 0 to 20, n is 0, 1, 2, or 3, A and B are individually selected from the group consisting of hydrogen, halogen, alkyl of 1 to 8 carbon atoms and aryl of 6 to 8 carbon atoms with the double bond geometry being E or Z or a mixture of E and Z or A and B form a third bond between the carbons to which they are attached, X_{A} is selected from the group consisting of alkyl, alkenyl, and alkynyl of 6 to 20 carbon atoms optionally interrupted with at least one heteroatom and optionally substituted with at least one halogen, cycloalkyl of 3 to 8 carbon atoms optionally substituted by a carbocyclic aryl, halogen, —CN, —OR₃, —COR₄, —COOR₅, —SR₆, —SOR₇, —SO₂R₈, 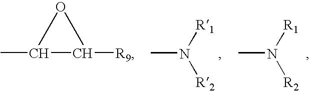
   —OC(Ar)₃, and a carbocyclic aryl and heterocyclic aryl optionally substituted, R₃, R₄, R₅, R₆, R₇, R₈, and R₉ are individually selected from the group consisting of hydrogen, alkyl of 1 to 8 carbon atoms optionally interrupted by at least one heteroatom and optionally substituted by at least one halogen, carbocyclic, and heterocyclic aryl and aralkyl of up to 14 carbon atoms optionally substituted with at least one member of the group consisting of free, salified, esterified, or amidified carboxy, —OH, halogen, —NO₂, —CN, alkyl, alkenyl, alkynyl, alkoxy, alkenyloxy, alkynyloxy, alkylthio, alkenylthio, alkynylthio, —SO-alkyl, —SO-alkenyl, —SO-alkynyl, —SO₂-alkyl, —SO₂alkenyl, and —S₂-alkynyl of up to 12 carbon atoms, all optionally substituted with at least one halogen, carbocyclic, and heterocyclic, aryl, O-aryl, and —S-aryl of up to 14 carbon atoms, R′₁ and R′₂ are individually selected from the group consisting of hydrogen and alkyl of 1 to 12 carbon atoms, R₁ and R₂ are individually selected from the group consisting of hydrogen, alkyl of 1 to 20 carbon atoms, —COAlk, and —COO-Alk, aryl, —CO-aryl, —COO-aryl, aralkyl, —CO-aralkyl, and —COO-aralkyl of up to 14 carbon atoms, Alk of alkyl of 1 to 8 carbon atoms, or R₁ and R₂ together with the nitrogen to which they are attached form ring of 3 to 8 members optionally containing a second heteroatom and optionally substituted with the above aryl substituents, Ar is a carbocyclic aryl optionally substituted with at least one of the above aryl substituents, Z is hydrogen or aryl of an organic carboxylic acid of up to 18 carbon atoms and their nontoxic, pharmaceutically acid addition salts.

In addition to the above compounds, additional macrolides and ketolides having an activity against TB were synthesized. The important factors considered when designing the present anti-TB macrolides and ketolides were: 1) structural optimization for potency of 14-membered macrolides, including modifications at the 6-, 9-, and 11-positions on the macrolactone, while avoiding toxic functionalities, and 2) potent anti-TB activity in order to shorten duration of the TB chemotherapy regimen.

Preliminary results showed that 3-cladinose-containing macrolides demonstrate a more potent anti-TB activity than their counterpart ketolides by one to two orders of magnitude. For example, a 4-[4-(3-pyridinyl)-1H-imidazol-1-yl]butyl group (1) substituent at the nitrogen atom in 11,12-carbamate of telithromycin and in RU69874, and the 3-(4-quinolinyl)propyl group (2) at 11,12-carbazate of RU66252 provided potent anti-TB drugs.

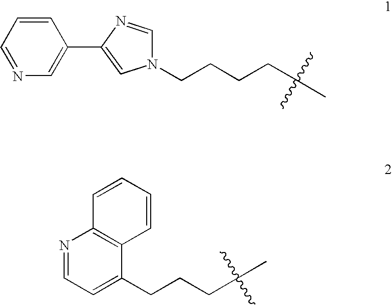

This and other discoveries led to a synthesis of 3-cladinose-containing macrolides of the corresponding ketolides having potent anti-Gram positive bacterial activities. Scale-up of the synthetic schemes set forth in Schemes 1-4 was performed for an in vivo study. The third step of the reaction sequences, i.e., the Michael addition in Scheme 1 and Eschweiler-Clarke-type reaction in Scheme 2, illustrate of points for facile diversification of molecular structure.

The following four macrolides were prepared, the syntheses of which are disclosed hereafter. Each compound, i.e., RU60887, RU66252, RU69874, and A323348, exhibited a very low minimum inhibitory concentration (MIC) vs. M. tuberculosis. In follow-up assays, RU66252 demonstrated an MIC vs. M. tuberculosis of about 9 μM, which is still an excellent MIC, and RU66252 also is active against M. tuberculosis in mice. In particular, RU66252 shows a dose response between 50 and 200 mg/kg in mice, with significant M. tuberculosis inhibition at 150 mg/kg and 200 mg/kg.

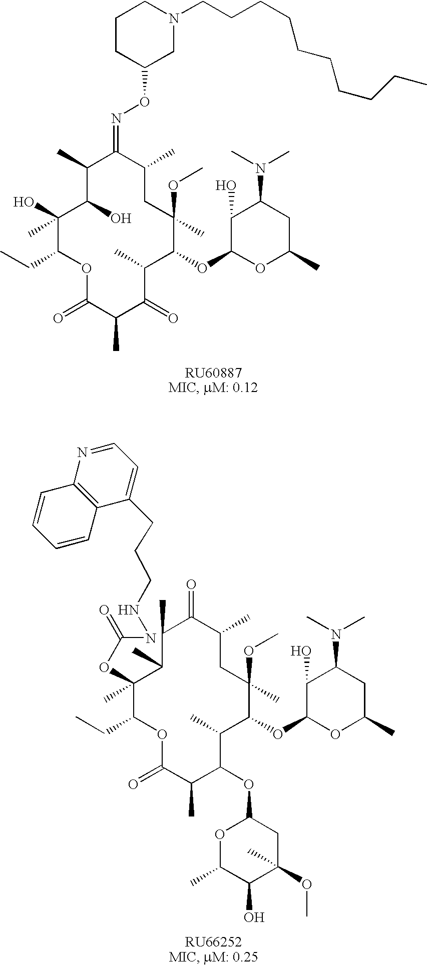

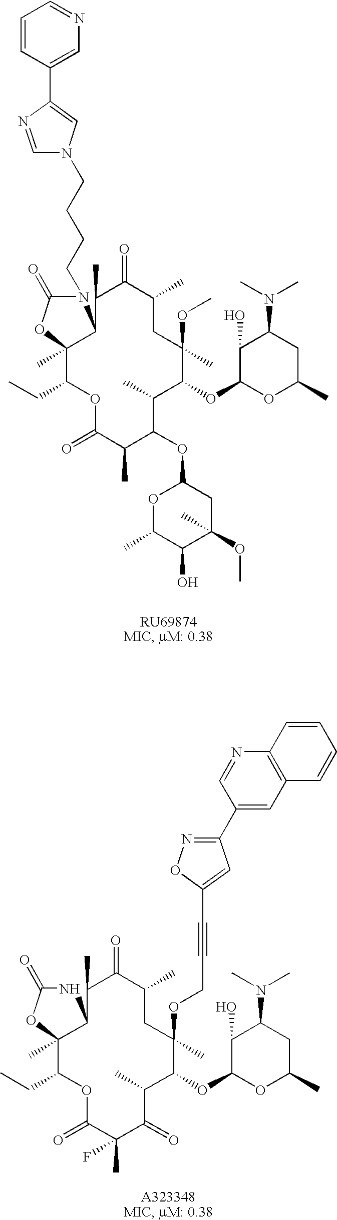

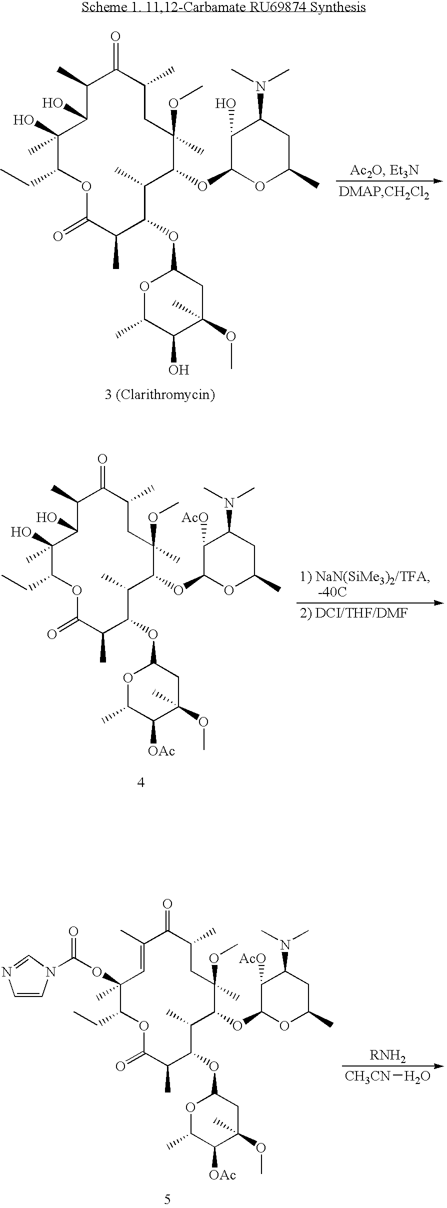

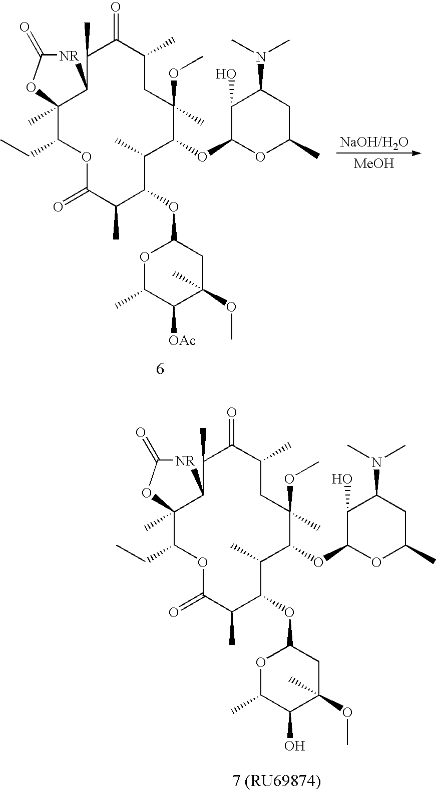

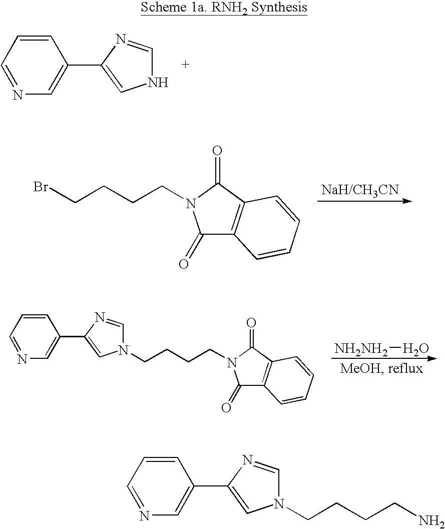

As discussed above, the four 11,12-carbamate ketolides, RU60856, RU62041, RU61143 and RU70332, demonstrated single digit MIC anti-TB activity with low toxicity in an in vitro study. Other amines that provide potent 11,12-carbamate anti-TB agents include: 4-(quinolinyl)butylamine (8), benzyloxyethylamine (9), 4-(3-chlorophenyl)-butylamine (10), 4-(8-methoxyquinolinyl)butylamine (11), 4-(6-methoxyquinolinyl)butylamine (12), 3-(aminophenyl)propylamine (13), 4-phenylbutylamine (14), and similar amines (15-25a).

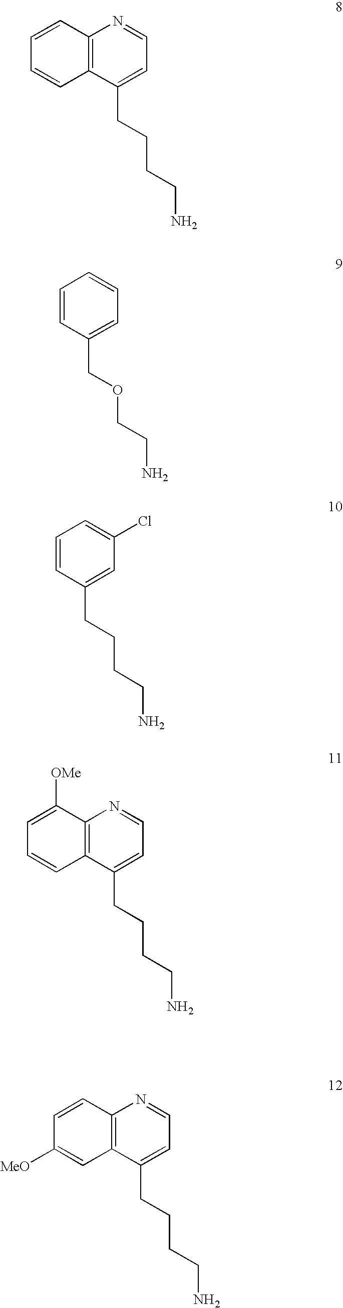

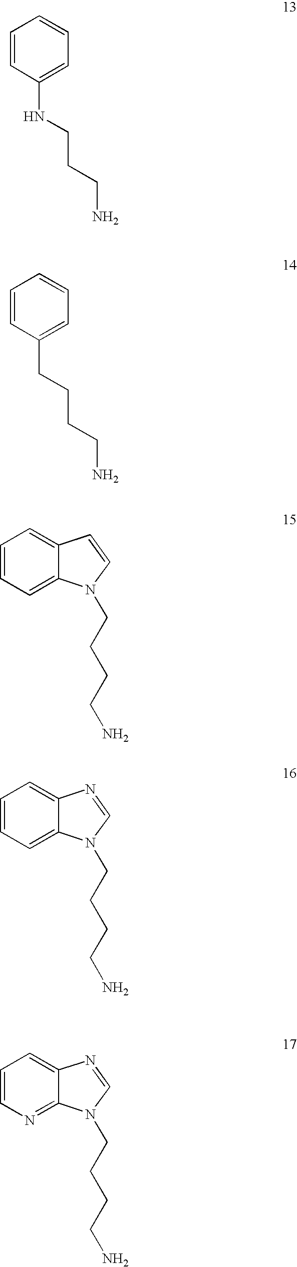

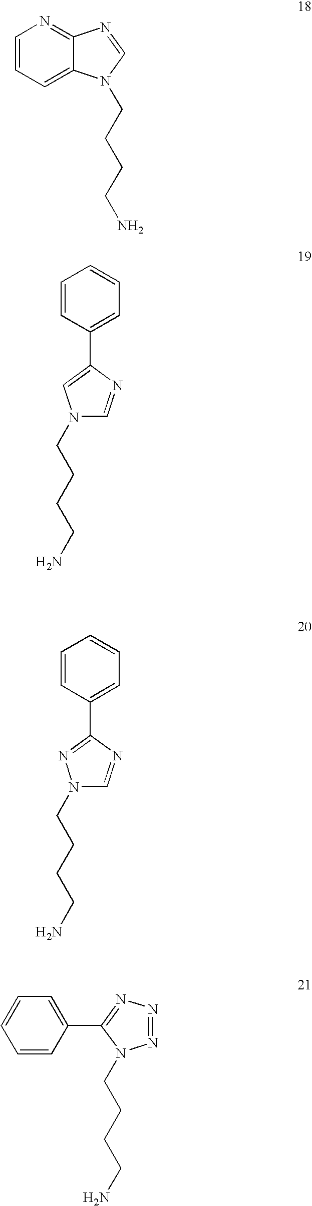

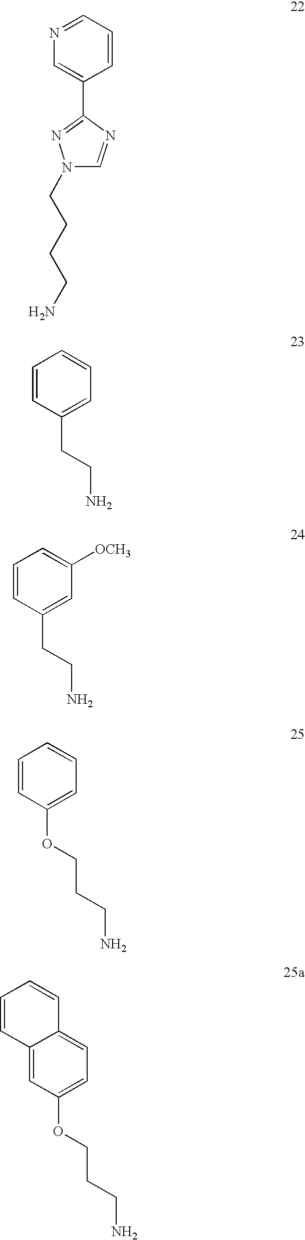

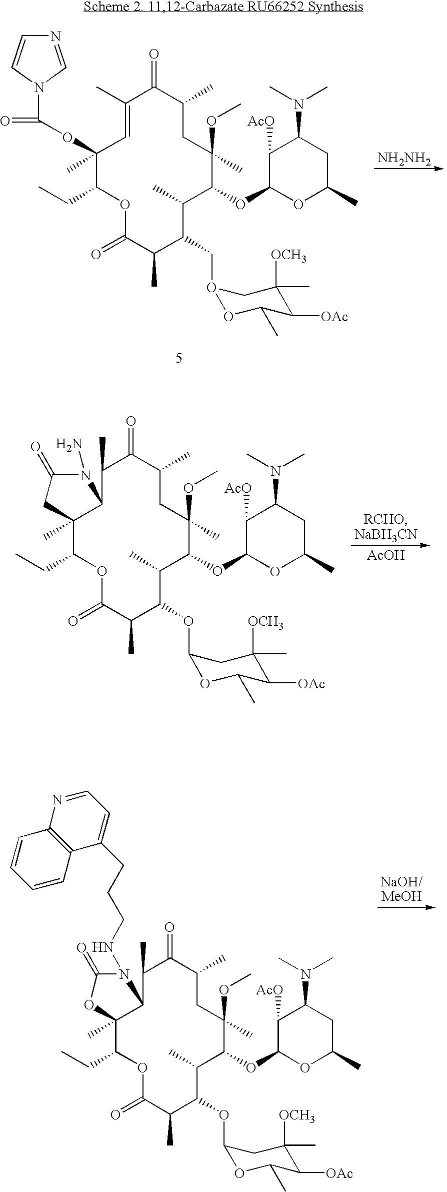

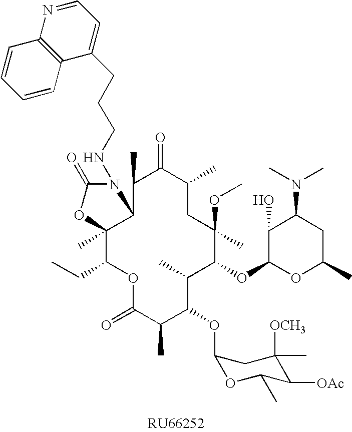

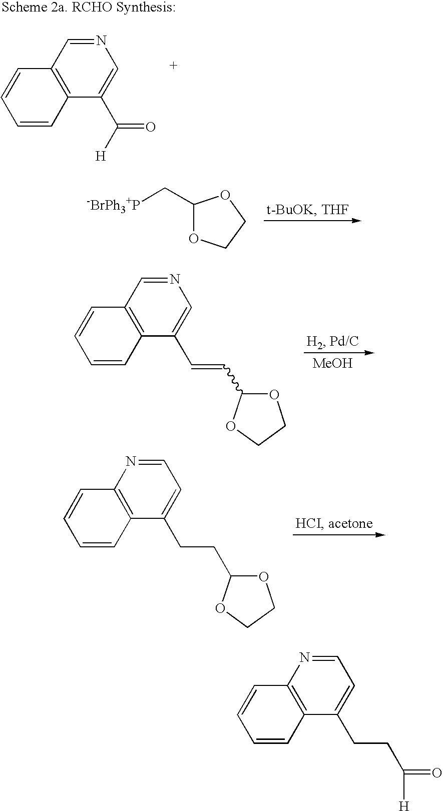

Following a synthetic route similar to Scheme 2, additional aldehydes can be used in the Eschweiler-Clarke-type reaction for parallel syntheses, including 3-(8-methoxyquinolinyl)propanal (26), 3-(6-methoxyquinolinyl)propanal (27), 3-(3-chorophenyl)propanal (28), benzenepropanal (29), and similar aldehydes, as set forth below.

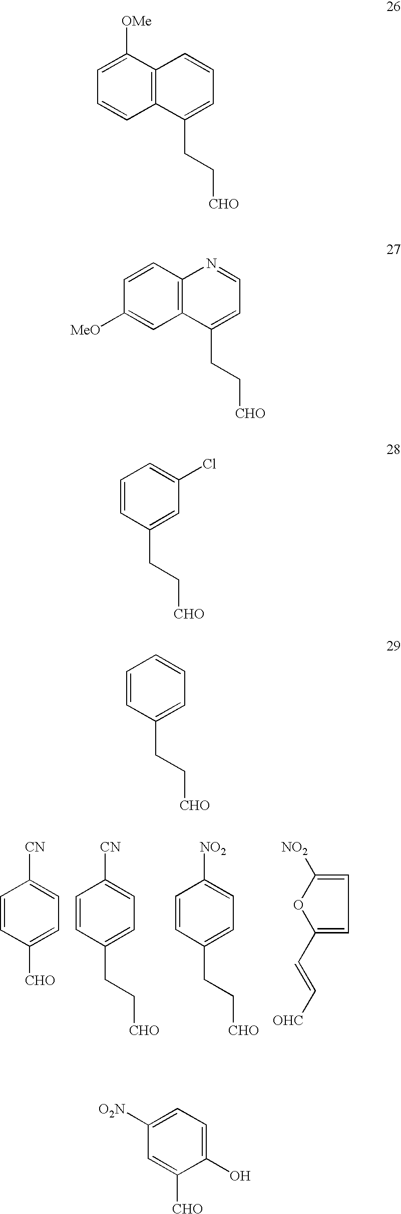

The carbazate analogue of RU69874 also can be synthesized by reacting compound 123 with compound 37, as set forth in Scheme 3.

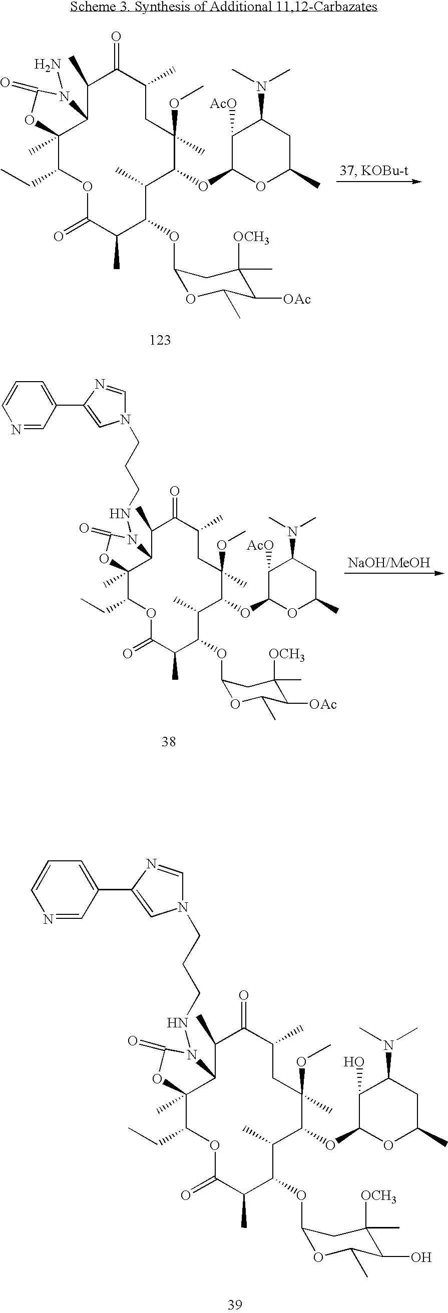

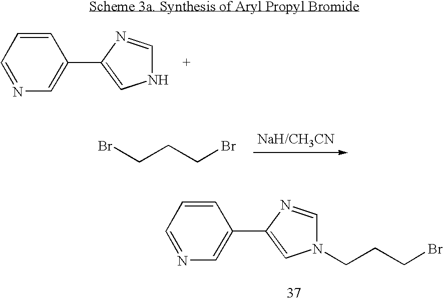

Additional aromatic heterocyles can be used for the syntheses shown in Scheme 3a.

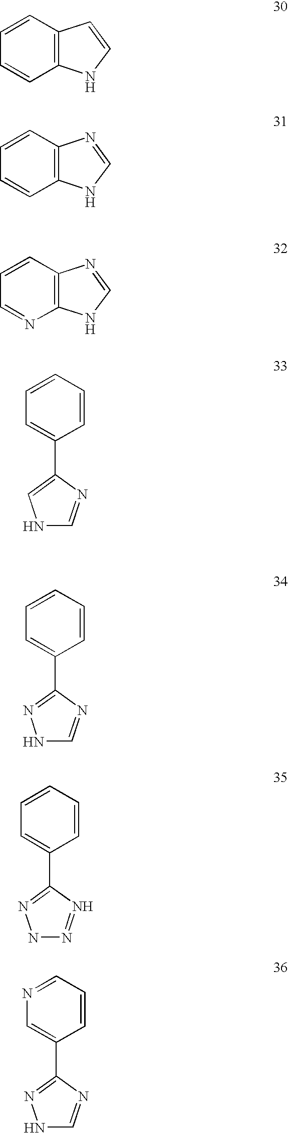

Analogous to the synthesis of 9-oxime ketolides (C. Agouridas et al., J. Med. Chem., 41:4080-4100 (1998)), 3-cladinose counterparts can be synthesized, as shown in Scheme 4. The synthetic route is shorter than ketolide synthesis because hydrolysis, protection, oxidation, and deprotection reactions can be omitted, while an improved opportunity to generate potent anti-TB compounds exists. The synthesis begins by coupling clarithromycin with a CBz protected hydroxylamine (Scheme 4a), followed by deprotection (41), and derivatization of the piperidine to the final 9-oxime 42.

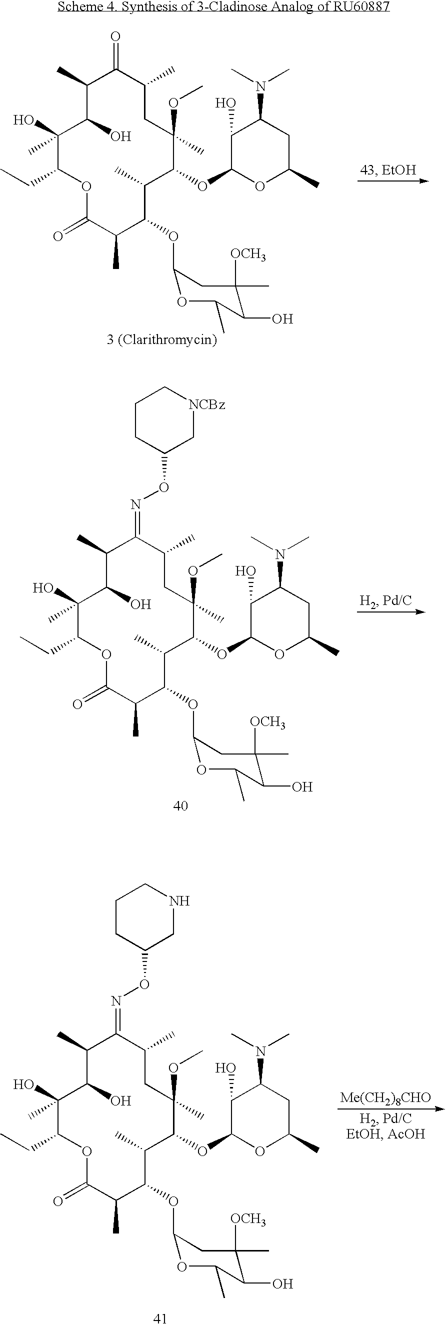

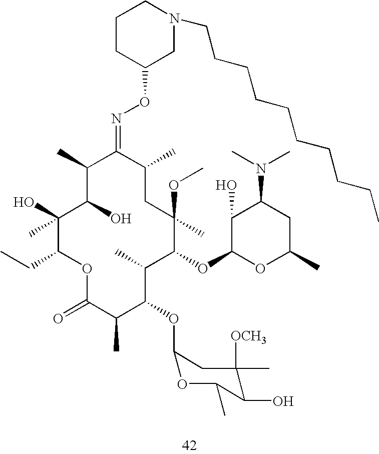

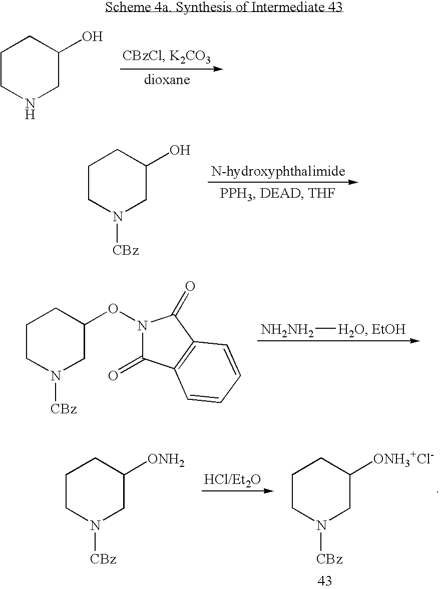

Additional aldehydes useful in the last step of Scheme 4 include, but are not limited to, 3-(4-hydroxyphenyl)propanal (44), 3-cyclohexylpropanal (45), formaldehyde (46), benzenepropanal (29).

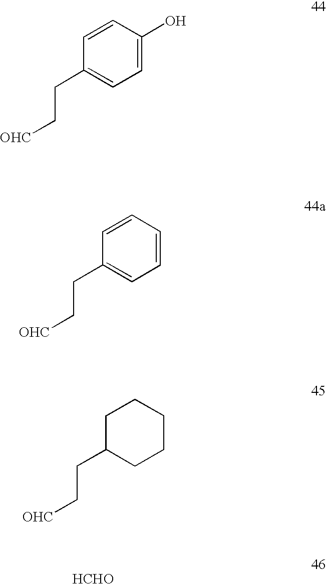

Additional hydroxylamines for use in the first step of Scheme 4 include, but are not limited to, hydroxylamine (47), ((2,4,6-trimethylphenyl)-methyl)hydroxylamine (48), (2-(dimethylamino)ethyl)-hydroxylamine (49). The hydroxylamines are used in their corresponding hydrochloride salt form. Only the first step in Scheme 4 is required to synthesize these 9-oximes

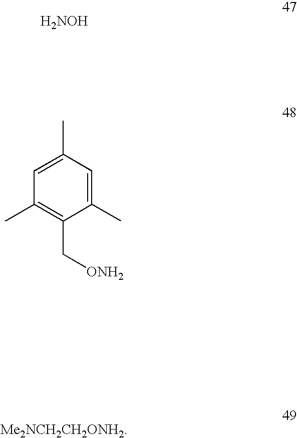

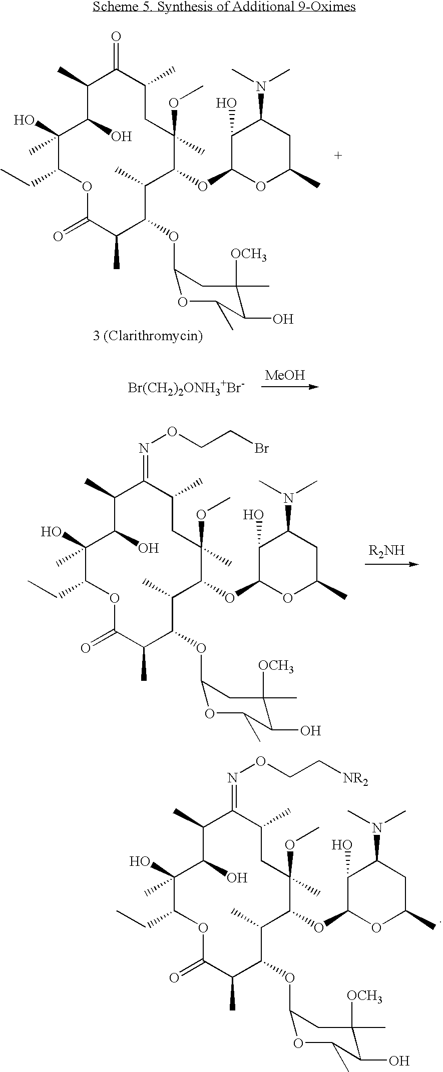

Amines useful for the final step of Scheme 5 include, but are not limited to, propylamine, 2-propynylamine (50), azetidine (51), 2-(1-pyrrolidinyl)ethylamine (52), and 3-(1H-imidazol-1-yl)-propylamine (53). 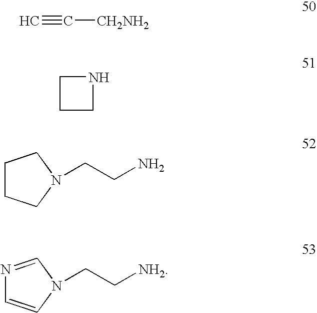
6-0-Functionalized 11,12-Carbamates

The synthesis of 6-0-aryl allyl carbamates begins with TMS (trimethylsilyl) protection on the 4″ and 2′ hydroxyl groups 55, followed by allylation of 55 to 56 (Z. Ma et al., J. Med. Chem., 44:4137-4156 (2001)). Treatment of 56 with NaHMDS and carbonyldiimidazole generates 57, followed by cyclization to carbamate 58. Heck coupling, followed by deprotection with TBAF, lead to final product 60.

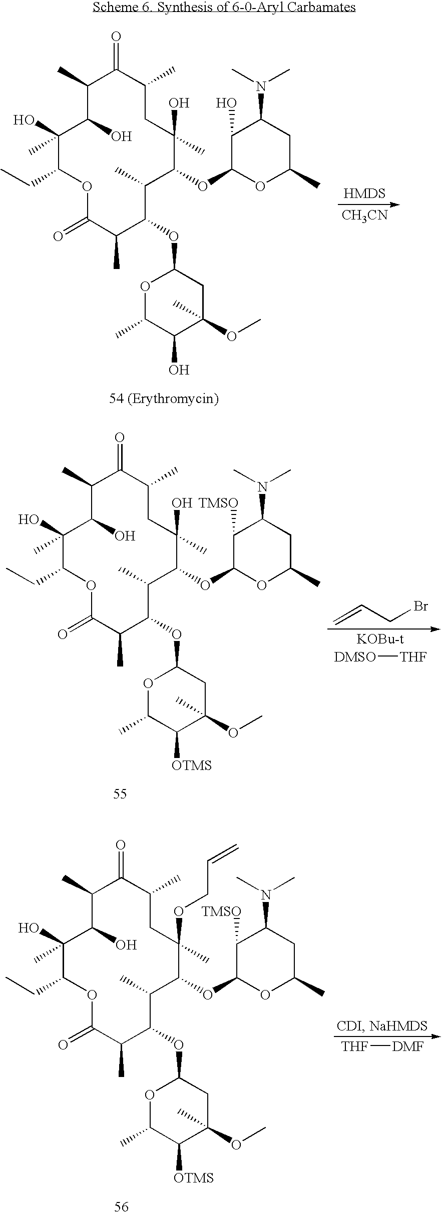

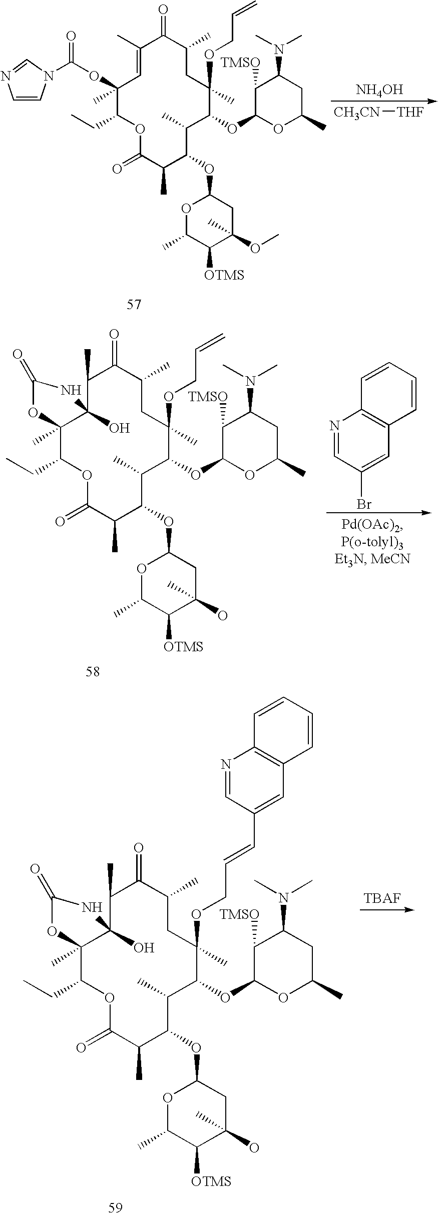

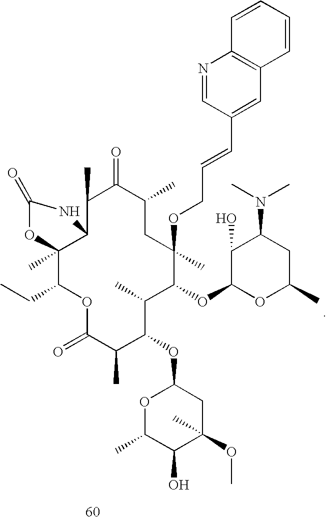

Additional aryl halides for use in the neck coupling reaction include, but are not limited to, 5-bromothieno[2,3b]pyridine (61), 7-bromoquinoline (62), 6-chloroquinoline (63), 3-bromo-1,8-naphthyridine (64), 3-bromo-1,6-naphthyridine (65), 3-bromo-1,5-naphthyridine (66), and 6-bromocinnoline (67).

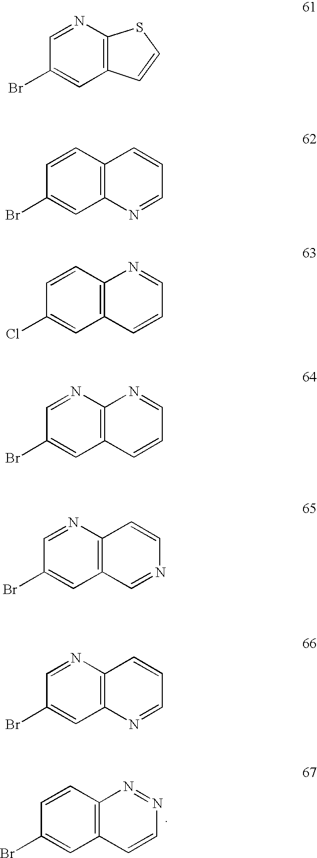

Scheme 7 illustrates a synthesis 6-0-aryl propargyl carbamates similar to the 6-0-aryl allyl carbamate in Scheme 6. The sythesis begins by propargylation of 155 (R. F. Clark et al., Bioorg. Med. Chem. Lett., 10:815-19 (2000)), followed by treatment with CDI. The acyl imidazole 154 is cyclized to carbamate 155, and subsequently coupled with an aryl halide (Scheme 7a) (L. T. Phan et al., Org. Lett., 2:2951-2954 (2000)). Deprotection provides the final product 157.

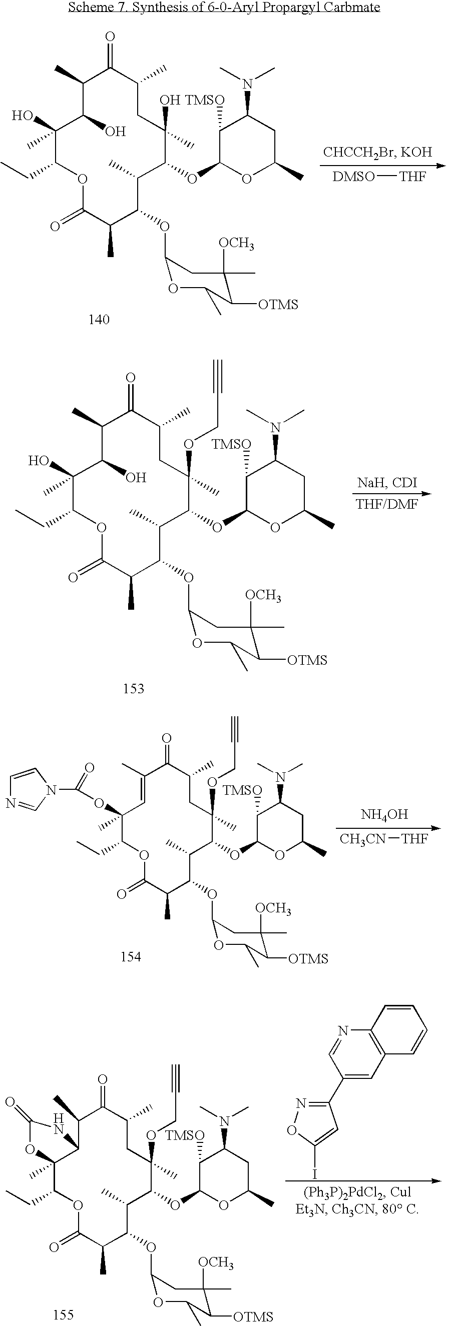

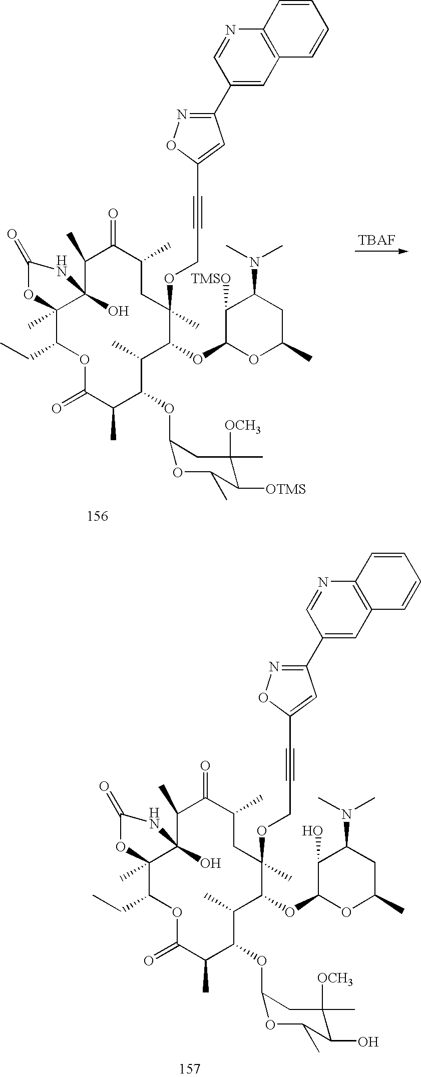

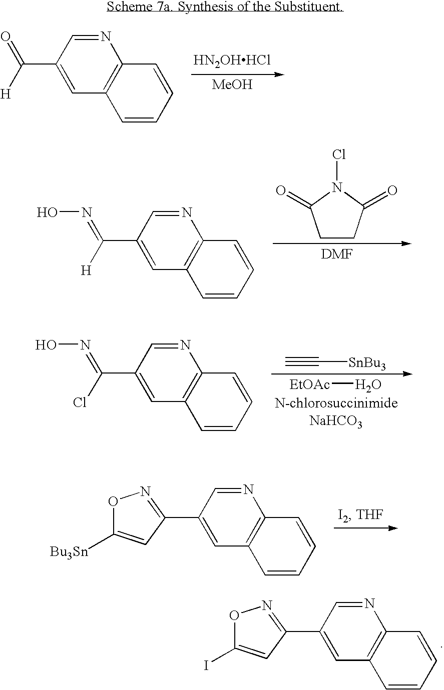

An additional aryl halides for use in Scheme 7 include, but are not limited to: 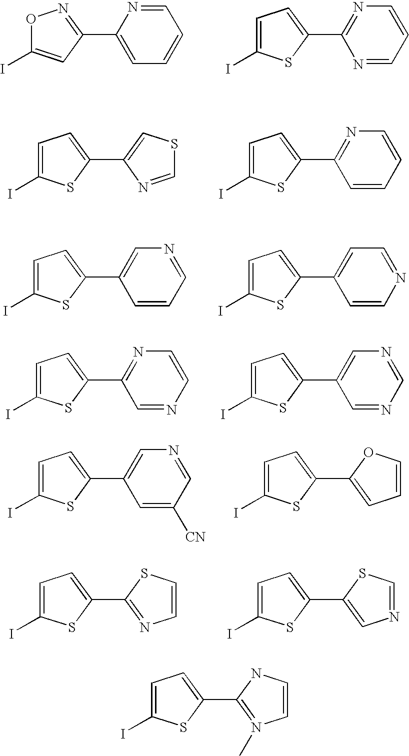
Azalides

Azalide analogues of the most active clarithromycin analogues, e.g., A323348, also can be synthesized. The synthesis begins with protection on reactive hydroxyl groups of azithromycin 158 to the protected form 159, which in turn couples with allyl bromide to 160. Heck coupling of 160 with the quinoline provides 161. TBAF deprotection provides to final product 162.

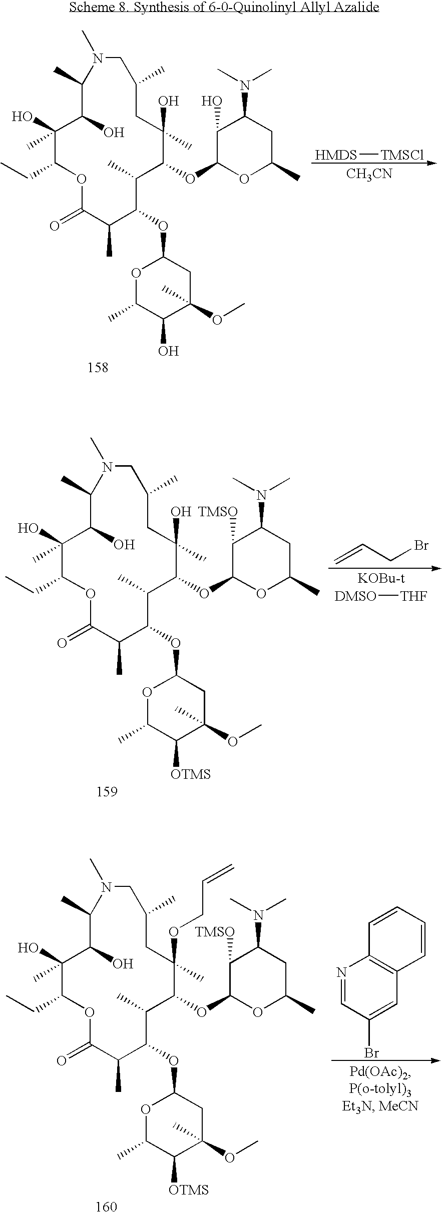

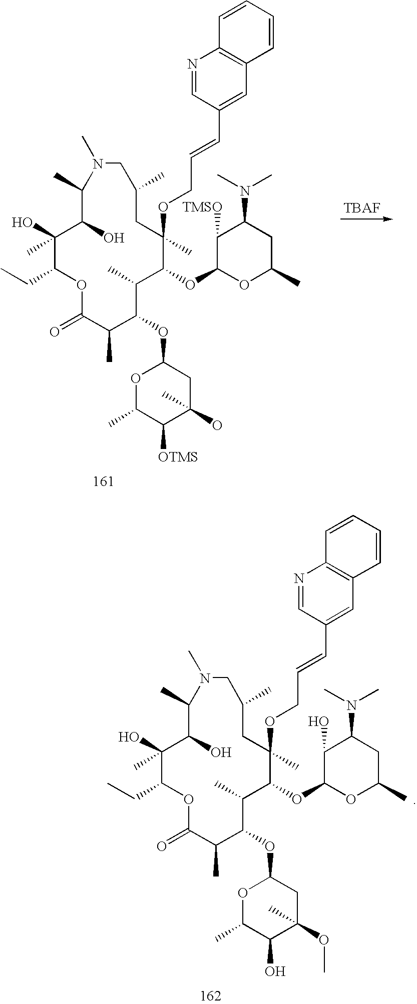

Synthesis of 6-0-aryl propargyl azalide begins with a common intermediate, the TMS protected azithromycin 159, with similar coupling methodology applied in Scheme 8, providing product 165.

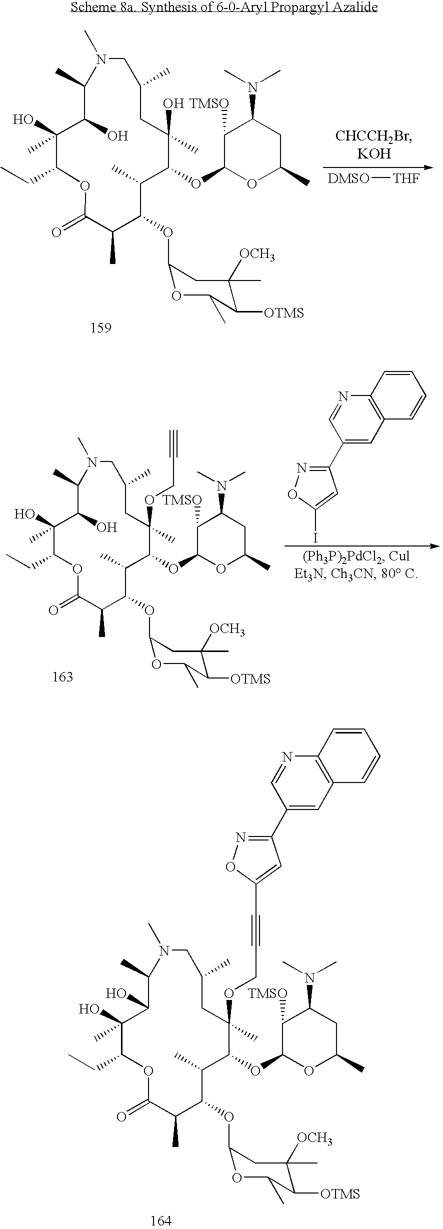

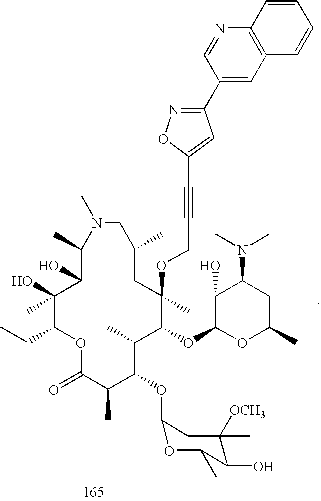

The macrolide or ketolide can be formulated to provide a pharmaceutical composition useful in a method of treating TB. The macrolide or ketolide active agent, or a mixture of active agents, typically is present in such a pharmaceutical composition in an amount of about 0.1% to about 75% by weight.

Pharmaceutical compositions containing a macrolide or ketolide, i.e., the active agents, are suitable for administration to humans or other mammals. Typically, the pharmaceutical compositions are sterile, and contain no toxic, carcinogenic, or mutagenic compound which would cause an adverse reaction when administered.

A pharmaceutical composition containing an active agent or mixture thereof can be administered by any suitable route, for example by oral, buccal, inhalation, sublingual, rectal, vaginal, intracisternal through lumbar puncture, transurethral, nasal, or parenteral (including intravenous, intramuscular, subcutaneous, and intracoronary) administration. A pharmaceutical composition containing the macrolide, ketolide, or mixture thereof preferably is administered by an oral or parenteral route. Parenteral administration can be accomplished using a needle and syringe. Implant pellets also can be used to administer an active agent parenterally. The active agents also can be administered as a component of an ophthalmic drug-delivery system.

The pharmaceutical compositions are administered in an effective amount to achieve its intended purpose. More specifically, a “therapeutically effective amount” means an amount effective to treat a disease. Determination of a therapeutically effective amount is well within the capability of those skilled in the art, especially in light of the detailed disclosure provided herein.

The exact formulation, route of administration, and dosage is determined by an individual physician in view of the patient's condition. Dosage amount and interval can be adjusted individually to provide levels of the active agents that are sufficient to maintain therapeutic or prophylactic effects.

The amount of pharmaceutical composition administered is dependent on the subject being treated, on the subject's weight, the severity of the affliction, the manner of administration, and the judgment of the prescribing physician.

Specifically, for administration to a human in the curative or prophylactic treatment of a disease, oral dosages of an active agent is about 10 to about 500 mg daily for an average adult patient (70 kg). Thus, for a typical adult patient, individual doses contain about 0.1 to about 500 mg active agent, in a suitable pharmaceutically acceptable vehicle or carrier, for administration in single or multiple doses, once or several times per day. Dosages for intravenous, buccal, or sublingual administration typically are about 0.1 to about 10 mg/kg per single dose as required. In practice, the physician determines the actual dosing regimen that is most suitable for an individual patient and disease, and the dosage varies with the age, weight, and response of the particular patient. The above dosages are exemplary of the average case, but there can be individual instances in which higher or lower dosages are merited, and such are within the scope of this invention.

An active agent can be administered alone, or in admixture with a pharmaceutical carrier selected with regard to the intended route of administration and standard pharmaceutical practice. Pharmaceutical compositions for use in accordance with the present invention, including ophthalmic preparations, thus can be formulated in a conventional manner using one or more physiologically acceptable carriers comprising excipients and auxiliaries that facilitate processing of an active agent into preparations that can be used pharmaceutically.

These pharmaceutical compositions can be manufactured in a conventional manner, e.g., by conventional mixing, dissolving, granulating, dragee-making, emulsifying, or lyophilizing processes. Proper formulation is dependent upon the route of administration chosen. When a therapeutically effective amount of an active agent is administered orally, the formulation typically is in the form of a tablet, capsule, powder, solution, or elixir. When administered in tablet form, the pharmaceutical composition additionally can contain a solid carrier, such as a gelatin or an adjuvant. The tablet, capsule, and powder contain about 5% to about 95%, preferably about 25% to about 90%, of an active agent of the present invention. When administered in liquid form, a liquid carrier, such as water, petroleum, or oils of animal or plant origin, can be added. The liquid form of the pharmaceutical composition can further contain physiological saline solution, dextrose or other saccharide solutions, or glycols. When administered in liquid form, the pharmaceutical composition contains about 0.5% to about 90%, by weight, of an active agent, and preferably about 1% to about 50%, by weight, of an active agent.

When a therapeutically effective amount of an active agent is administered by intravenous, cutaneous, or subcutaneous injection, the composition is in the form of a pyrogen-free, parenterally acceptable aqueous preparation. The preparation of such parenterally acceptable solutions, having due regard to pH, isotonicity, stability, and the like, is within the skill in the art. A preferred composition for intravenous, cutaneous, or subcutaneous injection typically contains an isotonic vehicle in addition to an active agent of the present invention.

An active agent can be readily combined with pharmaceutically acceptable carriers well-known in the art. Such carriers enable the active agent to be formulated as tablets, pills, dragees, capsules, liquids, gels, syrups, slurries, suspensions and the like, for oral ingestion by a patient to be treated. Pharmaceutical compositions for oral use can be obtained by adding the active agent with a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

An active agent can be formulated for parenteral administration by injection, e.g., by bolus injection or continuous infusion. Compositions for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous dispersions of the active agent. Additionally, suspensions of the active agent can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the dispersibility of the compounds and allow for the preparation of highly concentrated compositions. Alternatively, a present pharmaceutical composition can be in powder form for constitution with a suitable vehicle, e.g., sterile pyrogen-free water, before use.

An active agent also can be formulated in rectal compositions, such as suppositories or retention enemas, e.g., containing conventional suppository bases. In addition to the preparations described previously, an active agent also can be formulated as a depot preparation. Such long-acting preparations can be administered by implantation (for example, subcutaneously or intramuscularly) or by intramuscular injection. Thus, for example, an active agent can be formulated with suitable polymeric or hydrophobic materials (for example, as an emulsion in an acceptable oil) or ion exchange resins.

In particular, an active agent can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid compositions can be prepared with pharmaceutically acceptable additives, such as suspending agents. A composition also can be injected parenterally, for example, intravenously, intramuscularly, subcutaneously, or intracoronarily. For parenteral administration, the composition is best used in the form of a sterile aqueous solution which can contain other substances, for example, salts, or monosaccharides, such as mannitol or glucose, to make the solution isotonic with blood.

For veterinary use, an active agent is administered as a suitably acceptable formulation in accordance with normal veterinary practice. The veterinarian can readily determine the dosing regimen and route of administration that is most appropriate for a particular animal.

The present invention, therefore, discloses the use of a macrolide, a ketolide, or mixtures thereof, for the oral, parenteral, sublingual, rectal, vaginal, or urethral treatment of TB. The method comprises administering a therapeutically effective amount of a pharmaceutical preparation comprising an active agent.

Various macrolides and ketolides were evaluated in vitro against M. tuberculosis. For example, RU60887, RU66252, RU69874, and A323348 demonstrated a minimum inhibitory concentration (MIC) against M. tuberculosis of 0.12, 0.25, 0.38, and 0.38 μM, respectively. The discovery of submicromolar anti-TB MICs, and a cytotoxicity comparable to clarithromycin, of the four compounds was unexpected, as was the observation of an SAR that appeared unique to M. tuberculosis compared to an SAR previously observed with Staph and Strep.

In addition, telithromycin, cethromycin (ABT-773), and twenty-eight additional compounds were tested. Although highly active against Staph and Strep that are resistant to second-generation macrolides, telithromycin is even less active than clarithromycin against M. tuberculosis, confirming the previous observation made by Rastogi et al. (2000) for various mycobacteria. Although the cethromycin MIC of 3 uM against M. tuberculosis is superior to other commercial macrolides, it is still above the Cₘₐₓ, i.e., the maximum blood concentration, for erythromycin.

As illustrated in the following Tables 1, 1a, 2, and 2a, modifications on positions 6, 9, 11 and 12 of erythromycin provided compounds having a potent anti-TB activity and a low toxicity.

TABLE 1
The 11,12-Carbamate Derivatives Structures

| | Structure | | | | |
|---|---|---|---|---|---|
| | 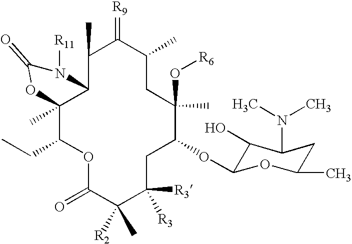 | | | | |
| Compound | R₂, | R₃, R₃′ | R₆, | R₉ | R₁₁ |
| RU66080 | H | 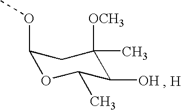 | Me | O | 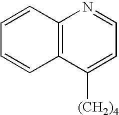 |
| RU66252 | H | 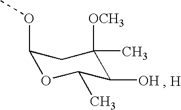 | Me | O | 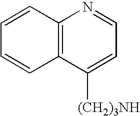 |
| RU004 | H | ═O | Me | O | 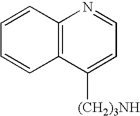 |
| RU3562 | F | ═O | Me | O | 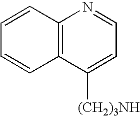 |
| RU69697 | H | ═O | Me | O | 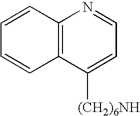 |
| RU69874 | H | 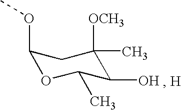 | Me | O | 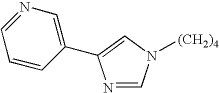 |
| Telithromycin | H | ═O | Me | O | 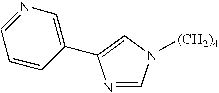 |
| RU60856 | H | ═O | Me | O | 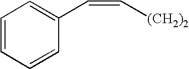 |
| RU61143 | H | ═O | Me | O | 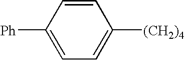 |
| RU63013 | H | ═O | Me | O | |
| RU66898 | H | ═O | Me | O | |
| RU62041 | H | ═O | Me | O | 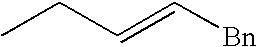 |
| RU62543 | H | ═O | Me | O | 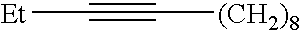 |
| RU60849 | H | ═O | Me | O | 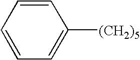 |
| RU70332 | H | ═O | Me | O | 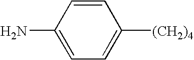 |
| RU70645 | H | ═O | Me | O | 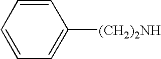 |
| RU61205 | H | ═O | Me | O | 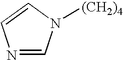 |
| RU66740 | H | ═O | Me | O | 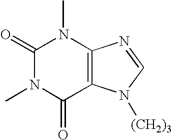 |
| A323348 | F | ═O | 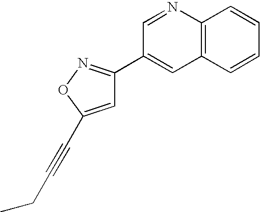 | O | H |
| ABT773 | H | ═O | 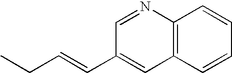 | O | H |
| A192803 | H | ═O | 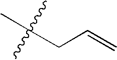 | O | H |

TABLE 1a
Anti-TB Activity (MIC, μM) and Toxicity (IC₅₀, μM)
of 11,12-Carbamate Derivatives

| | | Vero | | J774 | |
|---|---|---|---|---|---|
| Compound | MIC | IC₅₀ | SI | IC₅₀ | SI |
| RU66080 | 0.44 | 8.1 | 18.41 | 4.95 | 11.14 |
| RU66252 | 0.25 | 24.93 | 99.72 | 16.13 | 64.52 |
| RU004 | 4 | 26.34 | 6.58 | | |
| RU3562 | 16 | | | | |
| RU69697 | 4 | 7.32 | 1.83 | | |
| RU69874 | 0.38 | 26.96 | 70.95 | 58.59 | 154.18 |
| Telithromycin | 48 | | | 102.4 | 2.2 |
| RU60856 | 4 | >104.8 | >26.2 | 102.4 | 25.6 |
| RU61143 | 2 | 24.25 | 12.13 | 6 | 3 |
| RU63013 | 3 | 7.37 | 2.46 | | |
| RU66898 | 3.3 | 6.93 | 2.1 | | |
| RU62041 | 1 | 8.19 | 8.19 | 13.3 | 7.6 |
| RU62543 | 2.67 | 8.71 | 3.26 | | |
| RU60849 | 3 | 8.84 | 2.95 | | |
| RU70332 | 6 | 26.04 | 4.34 | | |
| RU70645 | 14 | | | | |
| RU61205 | 112 | | | | |
| RU66740 | >128 | | | | |
| A323348 | 0.38 | 24.29 | 63.92 | 15.27 | 40.18 |
| ABT773 | 3 | >104.8 | >34 | 102.4 | 34.1 |
| A192803 | >128 | | | | |
| RMP | 0.076 | 46.7 | 614 | 32.66 | 430 |

TABLE 2
The 11,12-Diol Derivatives Structures

| Compound | Structure | | |
|---|---|---|---|
| | 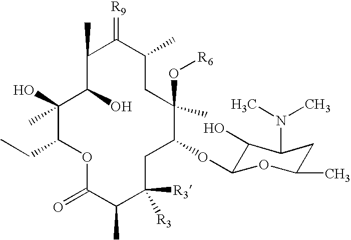 | | |
| | R₃, R₃′ | R₆ | R₉ |
| RU60887 | ═O | Me | 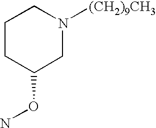 |
| RU61804 | ═O | Me | 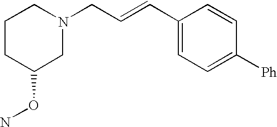 |
| ITR002 | ═O | Me | 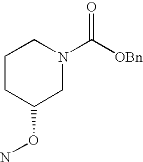 |
| ITR003 | ═O | Me | 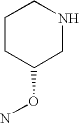 |
| RU54615 | ═O | Me | NOCH₂O(CH₂)₂OCH₃ |
| RU29558 | 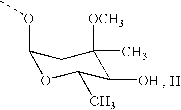 | H | NOCH₂OBn |
| RU56006 | ═O | Me | O |
| Clarithromycin | 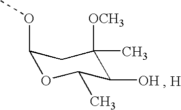 | Me | O |

TABLE 2a
Anti-TB Activity (MIC, μM) and Toxicity
(IC₅₀, μM) of the 11,12-Diols

| | |
|---|---|
| Vero | J774 |

| Compound | MIC | IC₅₀ | SI | IC₅₀ | SI |
|---|---|---|---|---|---|
| RU60887 | 0.125 | 27.58 | 220.64 | 4.7 | 37.6 |
| RU61804 | 0.5 | 9.12 | 18.24 | 3.9 | 7.8 |
| ITR002 | 64 | | | | |
| ITR003 | 128 | | | | |
| RU54615 | >128 | | | | |
| RU29558 | 7 | 28.15 | 4.02 | | |
| RU56006 | >128 | | | | |
| Clarithromycin | 8 | 40.99 | 5.12 | | |

The results summarized in Tables 1 and 2 illustrate some aspects of the anti-TB activity of macrolides. The most potent compound, RU60887, exhibited an MIC of 0.125 μM. It is envisioned that derivatives of RU60887 and other macrolides would provide more potent anti-TB compounds, for example, at least 10-fold or more potent MIC values vs. M. tuberculosis. Importantly, some of the most potent compounds also demonstrated a low toxicity. When VERO cells were used in a toxicity assay, RU60887 exhibited a selectivity index (SI) of 221. When more sensitive J774 cells were used, RU69874 displayed an SI of 154.

The data summarized in these tables show that in a comparison of three pairs of corresponding macrolides and ketolides, i.e., RU69874 and telithromycin, RU66252 and RU004, clarithromycin, and RU56006, each macrolide was significantly more potent than the counterpart ketolide. The enhancements in potency were 126, 16, and 16 fold, respectively. In addition, the macrolides not only exhibited an enhanced potencies over ketolides, but toxicities were not sacrificed to a significant degree. For example, when comparing (a) RU66252 to RU004 and (b) RU69874 to telithromycin, toxicity either was unchanged (25 μM vs. 26 μM, based on VERO cells) or changed insignificantly (59 μM vs. 102 μM, based on J774 cells). Accordingly, a macrolide is a preferred compound for use in the present method of treating TB.

Table 2 summarizes activities of the 9-oxime compounds. The two piperidine compounds, i.e., RU60887 and RU61804, are the most potent of the tested 9-oxime compounds. It was noted that a 9-oxime substitution alone did not provide potency, as shown from the results for RU54615.

Low Dose Aerosol Model of Acute Infection.

Prior to assessing in vivo efficacy, RU66252 and RU69874 were administered to pairs of female Balb/C mice once daily for 5-day cycles, and observed for overt signs of toxicity (e.g., weight loss, ruffled fur, and huddling), after which the mice were rested for 1 or 2 days. Then the dosage was increased for another 5-day cycle. Overall, the same mice received sequentially for 5 days: 200, 300, 400, and 500 mg/kg. No signs of overt toxicity were noted throughout the study.

Female BALB/c mice were infected via aerosol with a low dose of M. tuberculosis Erdman. Mice were treated once daily by gavage from day 10-30 post-infection. RU66252 and RU69874 were assessed at both 100 and 200 mg/kg qd. RU60887 was produced in sufficient quantity to test at a single dosage—100 mg/kg qd. Untreated controls did not reach levels typically achieved (10⁵-10⁶/mouse) with colony counts of less than 10 on individual plates. Normal colony counts were obtained on treated mouse lung homogenates, and the results strongly suggest dose-dependent activity of RU66252 (no colonies on 10⁻² dilution plates) against M. tuberculosis in the acute phase of infection, with less activity noted for the other tested compounds.

In Vitro Activity and Selectivity.

The present compounds are tested for MIC against M. tuberculosis H₃₇Rv in axenic medium and for cytotoxicity against VERO cells.

Cytotoxicity. Compounds are routinely tested for cytotoxicity in the ITR using VERO cells (C. L. Cantrell et al., J. Nat. Prod., 59:1131-36 (1996); G. C. Mangalindan et al., Planta Med., 66:364-5 (2000)). Macrolides are tested against VERO cells at concentrations less than or equal to 1% of the maximum achievable stock concentration. This results in a final DMSO concentration of less than or equal to 1% v/v, which is approximately the maximum non-cytotoxic concentration. Testing at very high concentrations allows for the recognition of high degrees of selectivity. Repeat testing is performed for compounds for which the IC₅₀ is less than or equal to the lowest tested concentration, when this concentration also is above the MIC for M. tuberculosis. After 72 hours exposure, viability is assessed on the basis of cellular conversion of MTS into a soluble formazan product using the Promega CellTiter 96 Aqueous Non-Radioactive Cell Proliferation Assay. Rifampin, clarithromycin, cethromycin, and telithromycin are included as controls.

For macrolides having an IC₅₀:MIC ratio greater than >10, cytotoxicity is repeated using the J774.1 macrophage cell line because these are used in the macrophage assay and typically all more sensitive than VERO cells.

Macrophage assay. Compounds for which the IC₅₀:MIC (SI) ratio is greater than >10 are tested for killing of M. tuberculosis Erdman (ATCC 35801) in monolayers of J774.1 murine macrophages (EC₉₉ and EC₉₀; lowest concentration effecting a 90% and 99% reduction in colony forming units at 7 days compared to drug-free controls) at 4-fold or 5-fold concentrations with the lowest concentration just below the MIC.

Description of Assays Demonstrating Whole-Cell Activity (MIC) against M. tuberculosis

MIC/MBC. Compounds are evaluated for MIC vs. M. tuberculosis H₃₇Rv using the microplate Alamar Blue assay (MABA) described in (L. Collins et al., Antimicrob. Agents Chemother., 41:1004-9 (1997)) except that 7H12 media, rather than 7H9+glycerol+casitone+OADC, is used. The use of this and other redox reagents, such as MTT, have shown excellent correlation with cfu-based and radiometric analyses of mycobacterial growth. The MIC is defined as the lowest concentration effecting a reduction in florescence (or luminescence) of 90% relative to controls. Isoniazid and rifampin are included as positive quality control compounds for each test, with expected MIC ranges of 0.025-0.1 and 0.06-0.125 ug/ml, respectively. MBCs are determined by subculture onto 7H11 agar just prior to addition of Alamar Blue and Tween 80 reagents to the test wells. The MBC is defined as the lowest concentration reducing cfu BY 99% relative to the zero time inoculum.

Several additional compounds were synthesized by a parallel synthesis and tested for an ability to control M. tuberculosis in vitro. The compounds were prepared as follows.

1) 2′,4″-Diacetyl Clarithromycin. The synthesis begins with acetyl protection on commercially available clarithromycin (Scheme 9). Clarithromycin (10 g, 13.4 mmol) was dissolved in anhydrous dichloromethane (48 mL) and cooled 0° C., followed by addition of triethylamine (5.2 mL, 37.4 mmol), DMAP (0.078 g, 0.67 mmol), and acetic anhydride (3.0 mL, 32.1 mmol). The reaction mixture was stirred at room temperature overnight.

Saturated aqueous ammonium chloride (40 mL) was added into the reaction mixture, which then was extracted with dichloromethane (2×40 mL). The aqueous phase was neutralized with saturated aqueous sodium hydrogen carbonate, and extracted with dichloromethane (2×40 mL). The combined organic phase was dried over sodium sulfate. Filtration of sodium sulfate followed by evaporation of solvent, afforded diacetylated clarithromycin 1 as white powder (11.09 g, 13.3 mmol, 100%). Selected ¹H NMR (δ, CDCl₃, 300 MHz) resonance: 2.10, 2.06.

2) Acyl Imidazole 2. Protected clarithromycin 1 (1.66 g, 2 mmol) was dissolved in anhydrous THF (17 mL), cooled to −40° C., followed by addition of 1M THF solution of NaHMDS (2.4 mL, 2.4 mmol), and stirred for 40 min.

In a separate round-bottomed flask, CDI (1.3 g, 8 mmol) was dissolved in THF/DMF mixture (12 mL/8 mL), and transferred into the solution of 1, and stirred for 24 h at room temperature.

Ethyl acetate was added into the reaction mixture, followed by 5% aqueous sodium hydrogencarbonate solution. After separation of the phases, the organic phase was washed with brine. Drying over sodium sulfate and concentration afforded acyl imidazole 2 as solid foam (1.50 g, 1.65 mmol, 83%). Selected ¹H NMR (δ, CDCl₃, 300 MHz) resonance: 8.08, 7.36, 7.07, and 6.66.

3) Clarithromycin Carbazate 3. The acyl imidazole 2 (3.63 g, 4 mmol) was dissolved in acetonitrile (40 mL), followed by addition of hydrazine (1.95 mL, 40 mmol) and water (4.35 mL). The reaction mixture was stirred for 6 h at 60° C.

After concentration, the reaction mixture was purified with flash chromatography, providing the desired carbazate 3 as white powder with quantitative yield. Selected ¹³C NMR (δ, CDCl₃, 75 MHz) resonance: 217.5.

4) Library of 48 Functionalized Carbazates. Carbazate 3 (4.15 g, 50 mmol) was dissolved in anhydrous methanol (37.5 mL). This solution (0.75 mL, 0.1 mmol) was added into each of 48 reaction tubes in a Bohdan MiniBlock, followed by addition of 48 individual aldehydes in Chart 1, and glacial acetic acid (0.025 mL,0.4 mmol each). The reaction block was shaken on a vertex shaker for 20 h. Sodium cyanoboronhydride (0.2 mL, 0.2 mmol each of 1 M THF solution) was added into individual reaction tube, shaken for 4 h.

The 48 reaction mixtures were purified with SPE with a second MiniBlock. The reaction mixtures were loaded on 48 C-18 SPE cartridges, followed by water wash, and eluted into 48 collection tubes with methanol. After drying in a SpeedVac, powdery desired library 4 was obtained.

5) Deacetylation. Library 4 was dissolved in methanol (2 mL each), followed by addition of 2 N sodium hydroxide (0.3 mL each), and shaken for 24 h.

The reaction mixtures were purified with SPE in a similar fashion, obtaining the desired library 5 (4-50 mg) as powders. The identity and purity were assessed with LC-MS.

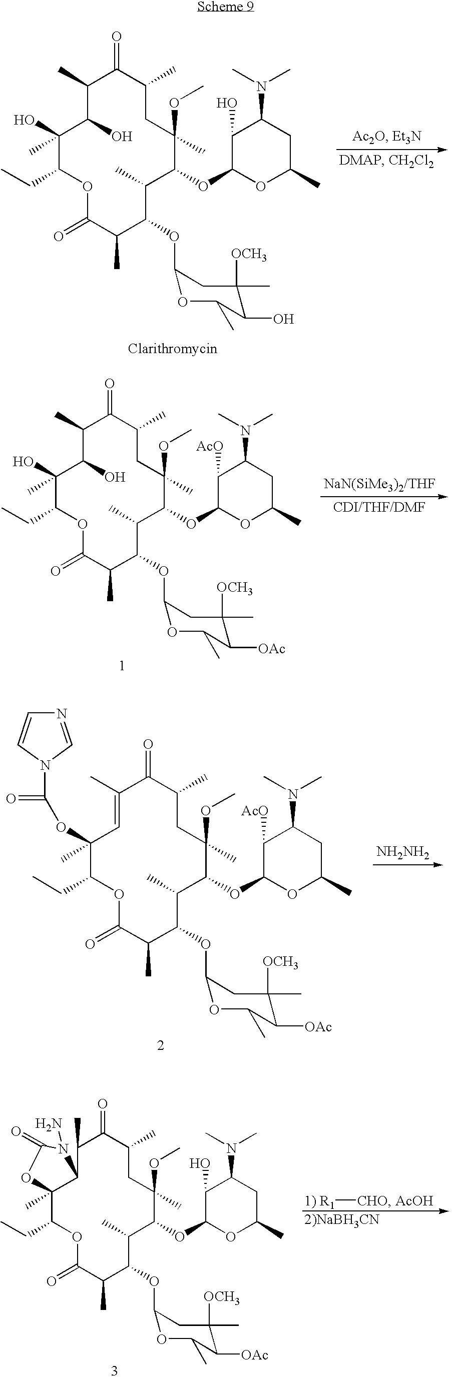

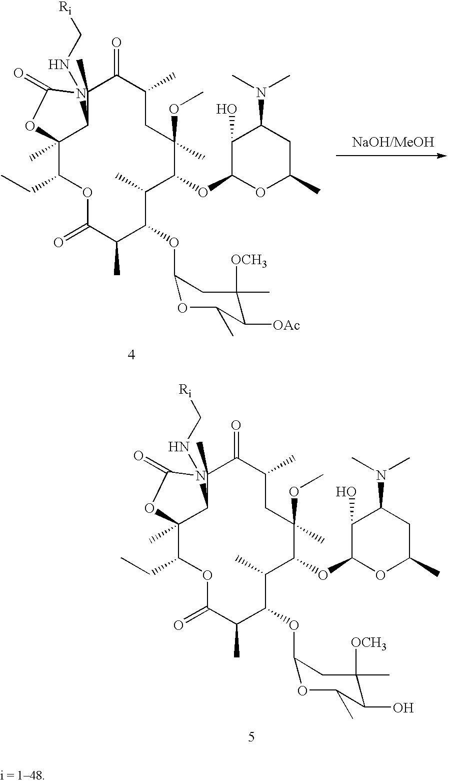

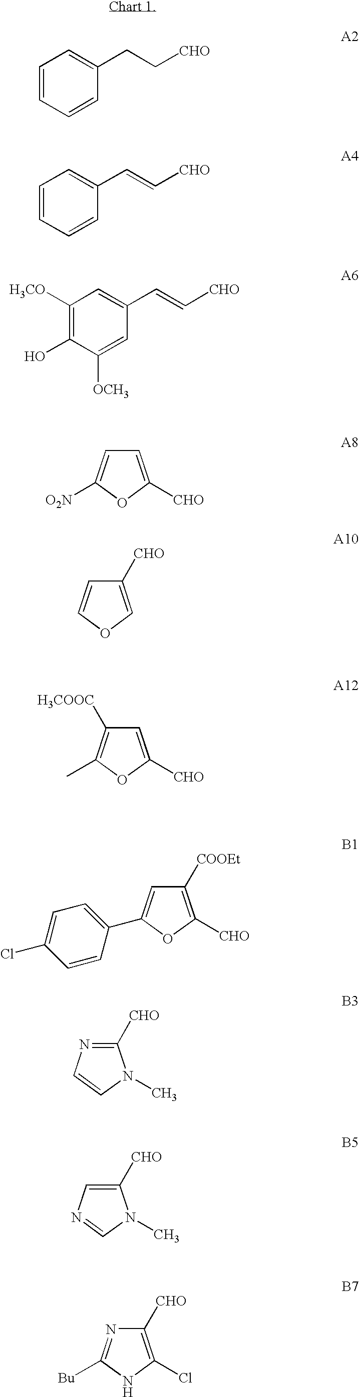

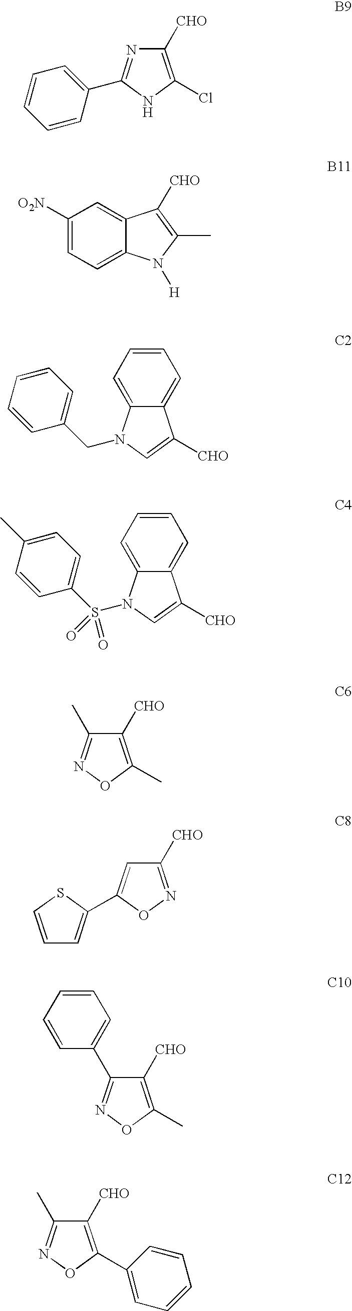

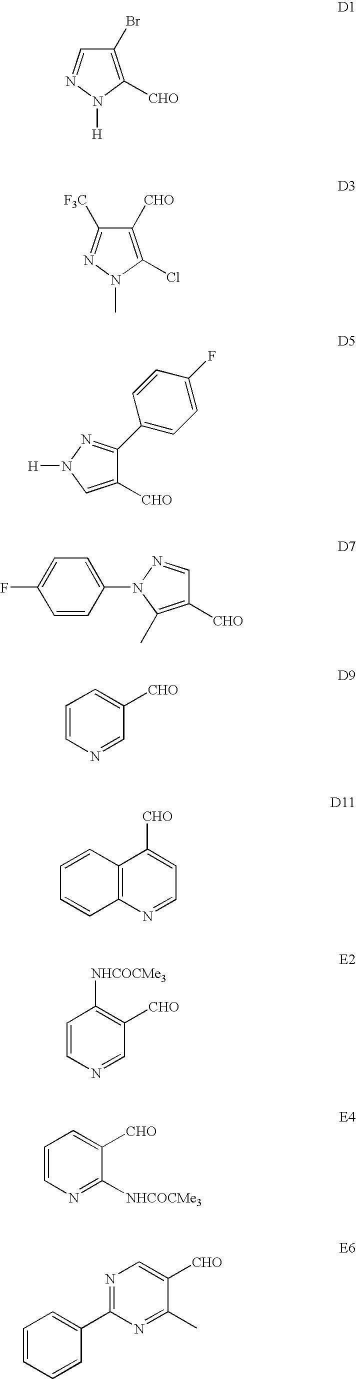

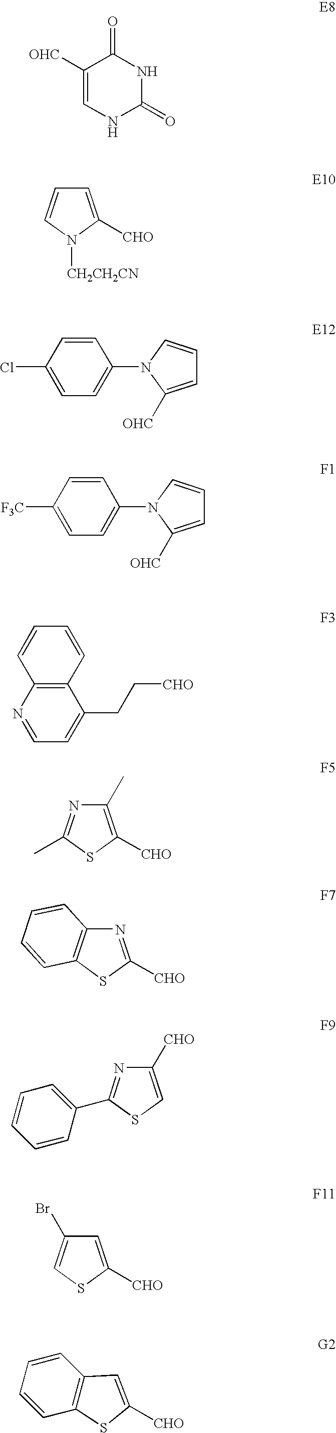

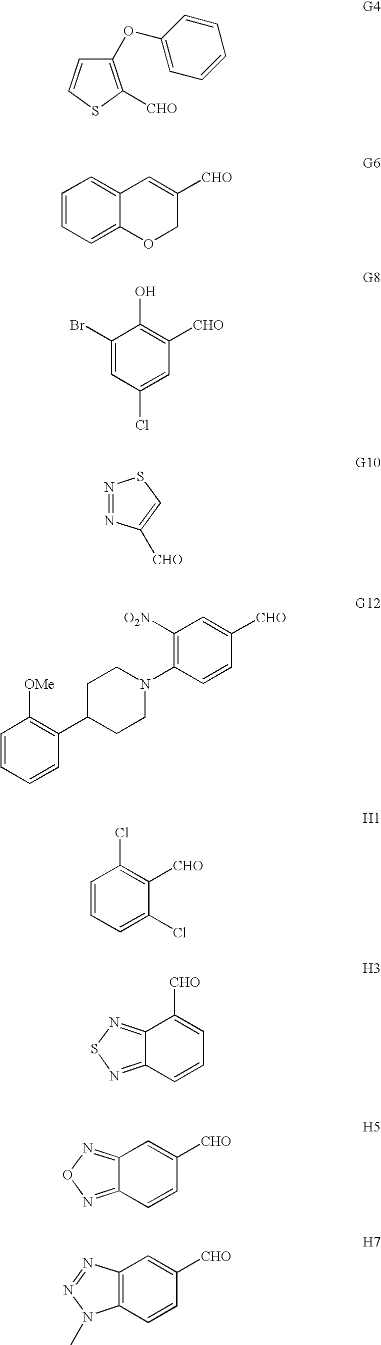

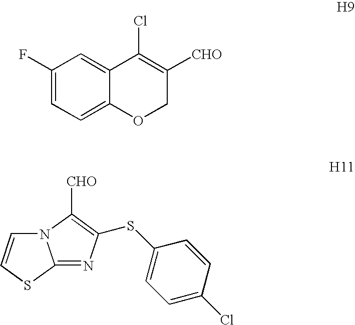

The compounds demonstrated the following MICs:

TABLE 3
MIC

| Compound | Prod.FW | MIC, μM |
|---|---|---|
| A2′ | 906.2 | 10 |
| A4′ | 904.1 | 7 |
| A6′ | 980.2 | 10.2 |
| A8′ | 913.1 | >9 |
| A10′ | 868.1 | >7 |
| A12′ | 940.1 | >12 |
| B1′ | 1050.7 | >18 |
| B3′ | 882.1 | >18 |
| B5′ | 882.1 | >27 |
| B7′ | 958.6 | >19 |
| B9′ | 944.2 | >14 |
| B11′ | 976.2 | >22 |
| C2′ | 1007.3 | 7 |
| C4′ | 1071.3 | 11 |
| C6′ | 897.1 | >17 |
| C8′ | 951.2 | ?9 |
| C10′ | 959.2 | 12 |
| C12′ | 959.2 | 9 |
| D1′ | 947.0 | 23 |
| D3′ | 984.5 | 20 |
| D5′ | 962.2 | 25 |
| D7′ | 976.2 | 13 |
| D9′ | 879.1 | >15 |
| D11′ | 929.1 | 16 |
| E2′ | 978.2 | >27 |
| E4′ | 978.2 | >27 |
| E6′ | 970.2 | 5 |
| E8′ | 912.1 | >20 |
| E10′ | 920.1 | 20 |
| E12′ | 977.6 | 3.4 |
| F1′ | 1011.2 | 17.6 |
| F3′ | 957.2 | 9 |
| F5′ | 913.2 | >19 |
| F7′ | 935.2 | 16 |
| F9′ | 961.2 | 12.6 |
| F11′ | 963.0 | 12.7 |
| G2′ | 934.2 | 11 |
| G4′ | 976.2 | 13 |
| G6′ | 932.1 | 11 |
| G8′ | 1007.4 | 14 |
| G10′ | 886.1 | >16 |
| G12′ | 1112.4 | 19 |
| H1′ | 947.0 | 11 |
| H3′ | 936.2 | >22 |
| H5′ | 920.1 | >20 |
| H7′ | 933.1 | >22 |
| H9′ | 984.6 | ?3 |
| H11′ | 1066.8 | 35.4 |

Despite the complexion of the reaction products, F3′ (the product from F3) is the desired product RU66252. Using RU66252 as a reference point, preferred compounds are shown in Chart 2.

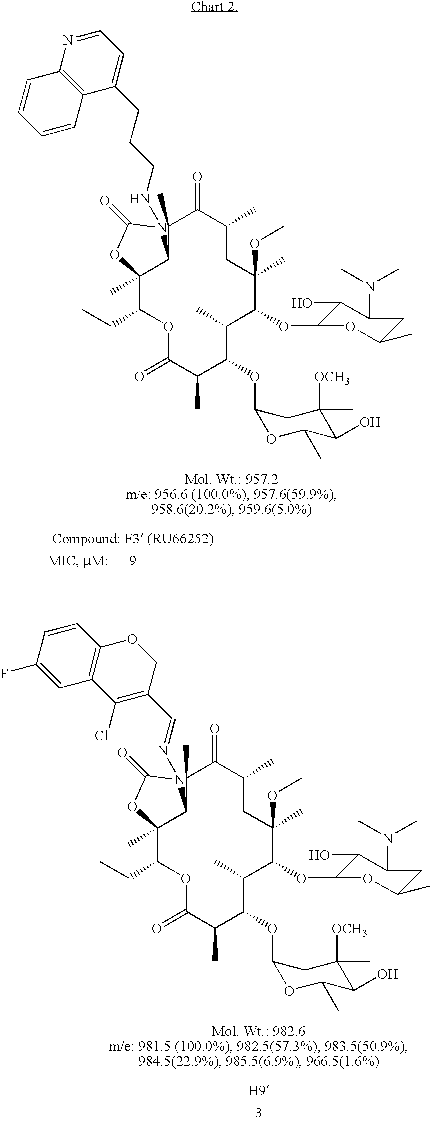

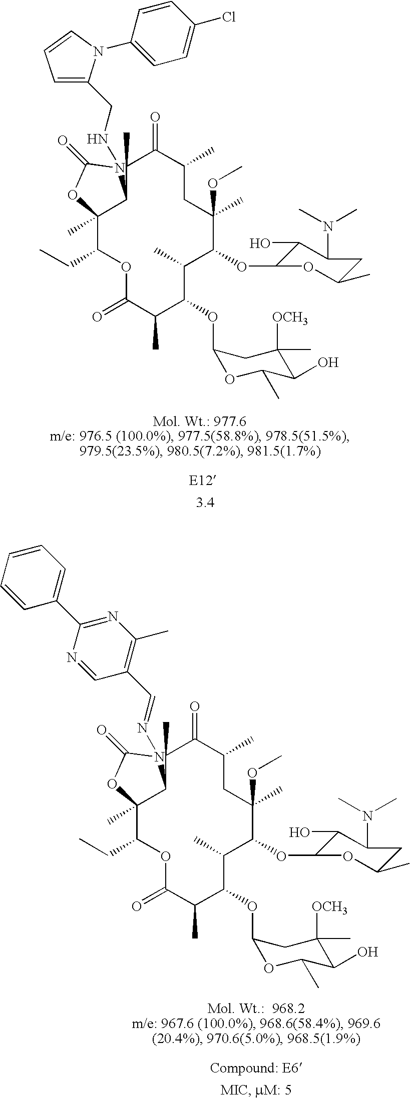

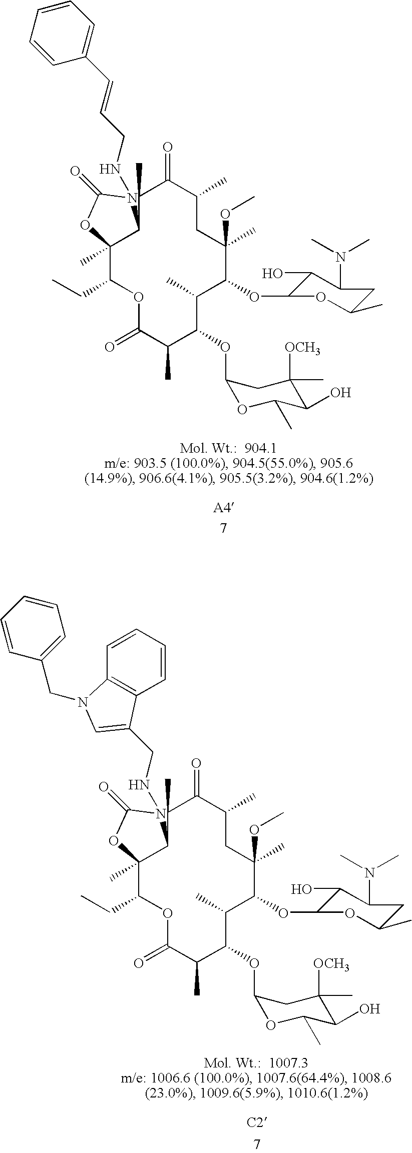

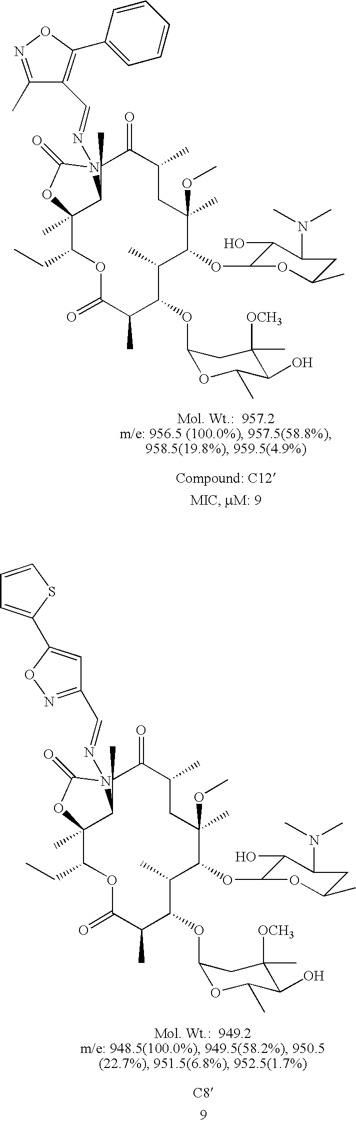

Modifications and variations of the invention as hereinbefore set forth can be made without departing from the spirit and scope thereof, and only such limitations should be imposed as are indicated by the appended claims.

1. A method of treating tuberculosis comprising administering a therapeutically effective amount of a macrolide, a ketolide, or a mixture thereof, to an individual in need thereof, wherein the macrolide or ketolide has an MIC vs. M. tuberculosis of about 50 μM or less.

2. The methodaim 1 wherein the macrolide or ketolide is disclosed in Table 1 of the specification.

3. The method of claim 1 wherein the macrolide or ketolide is disclosed in Chart 1 of the specification.

4. The method of claim 1 wherein the macrolide or ketolide is disclosed in Chart 2 of the specification.

5. The method of claim 1 wherein the macrolide or ketolide is selected from the group consisting of RU60887, RU66252, RU69874, RU60856, RU62041, RU61143, RU70332, A323348, and mixtures thereof.

6. The method of claim 1 wherein the macrolide or ketolide has a structural formula 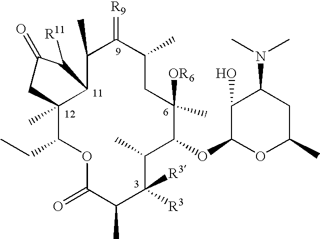
wherein R³ and R^{3 ′}are taken together as ═O, or R^{3′}is H and R³ is 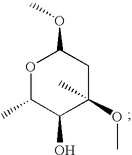
R⁶ is selected from the group consisting of: —CH₂—CH═CH—R^{a} and —CH₂C═C—R^{a}, wherein R^{a} is selected from the group consisting of: 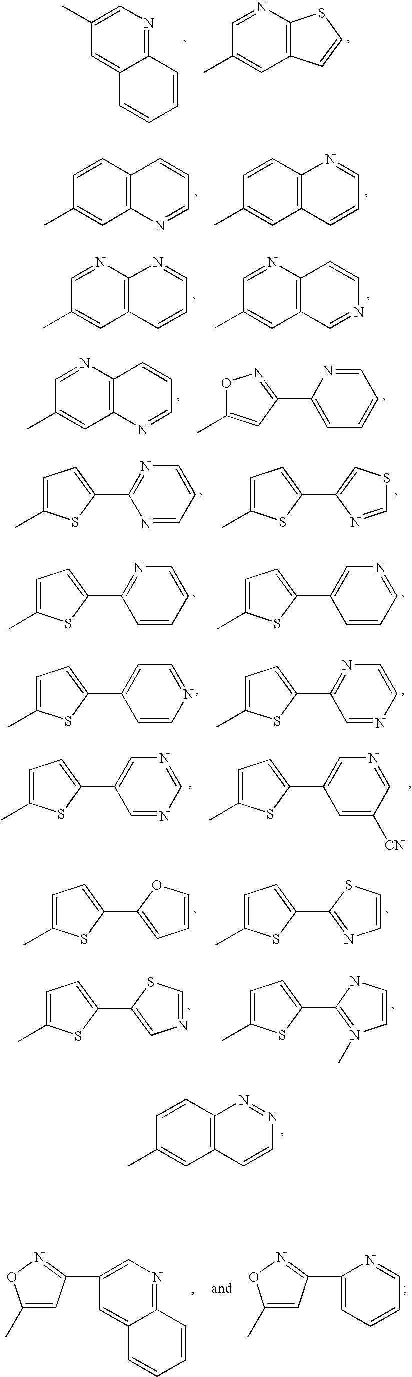
R⁹ is selected from the group consisting of: 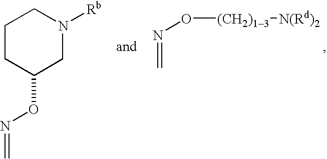
wherein R^{b} is selected from the group consisting of C₁₋₁₀ alkyl, 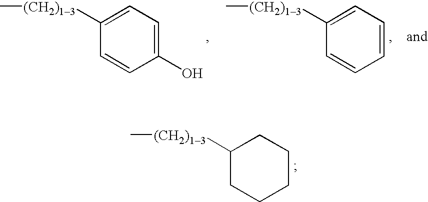
and R^{d} is selected from the group consisting of H, —CH₂—C≡CH, 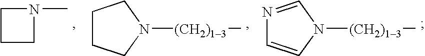
R¹¹ is selected from the group consisting of 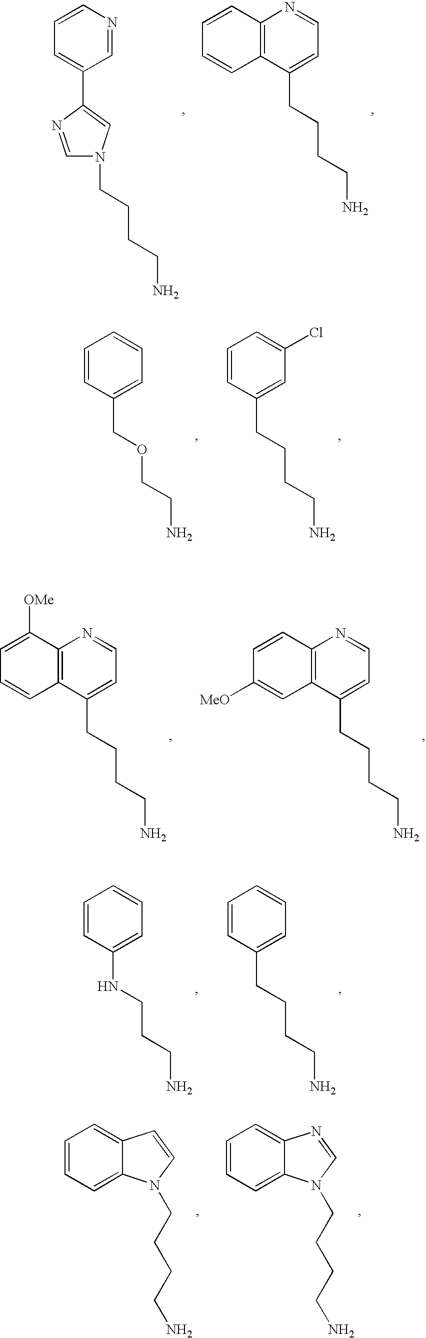 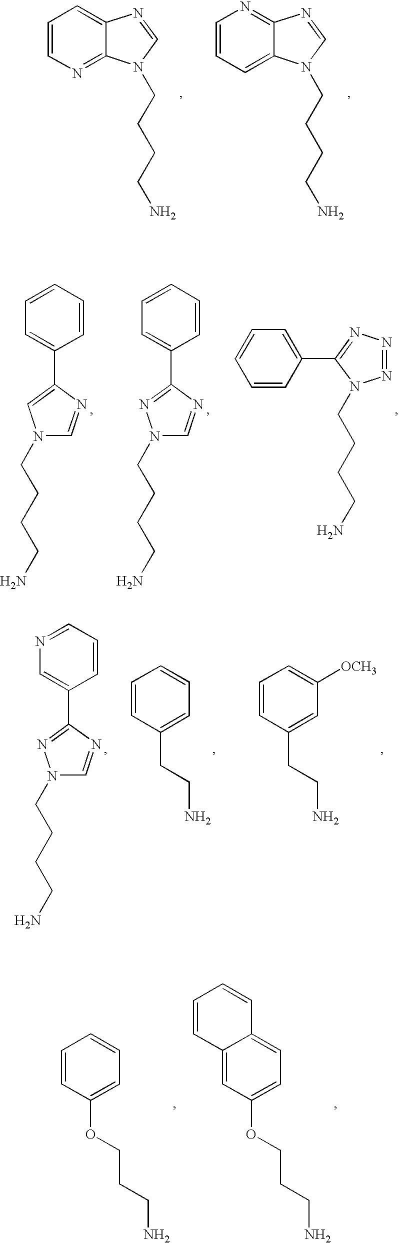
and —NHR^{c}, wherein R^{c} is selected from the group consisting of

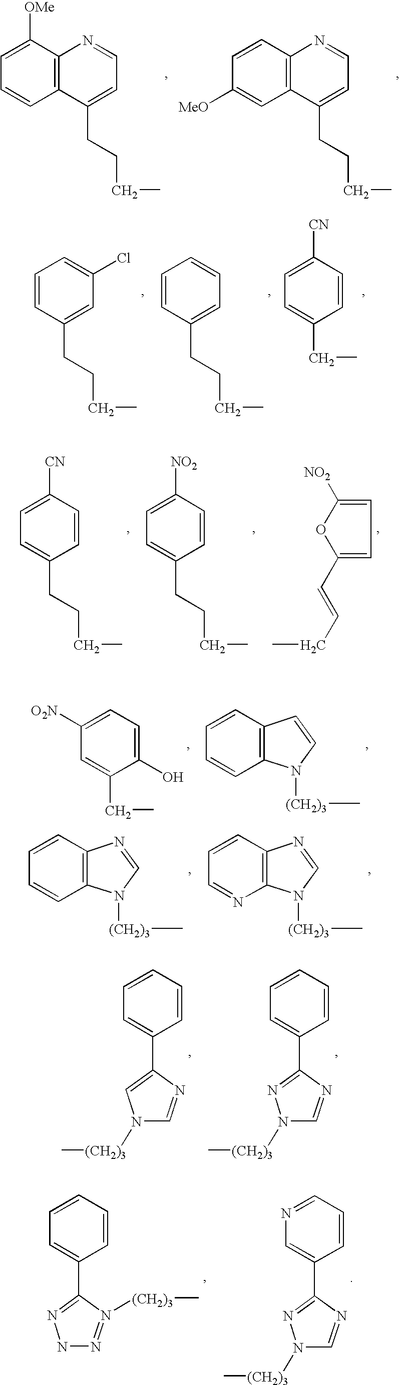

7. The method of claim 1 wherein the macrolide or ketolide has a structural formula 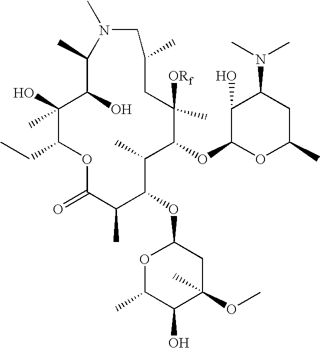
wherein R_{f} is selected from the group consisting of —CH₂CH═CH—R_{g} and —CH₂C≡C—R_{g}, wherein R_{g} is selected from the group consisting of

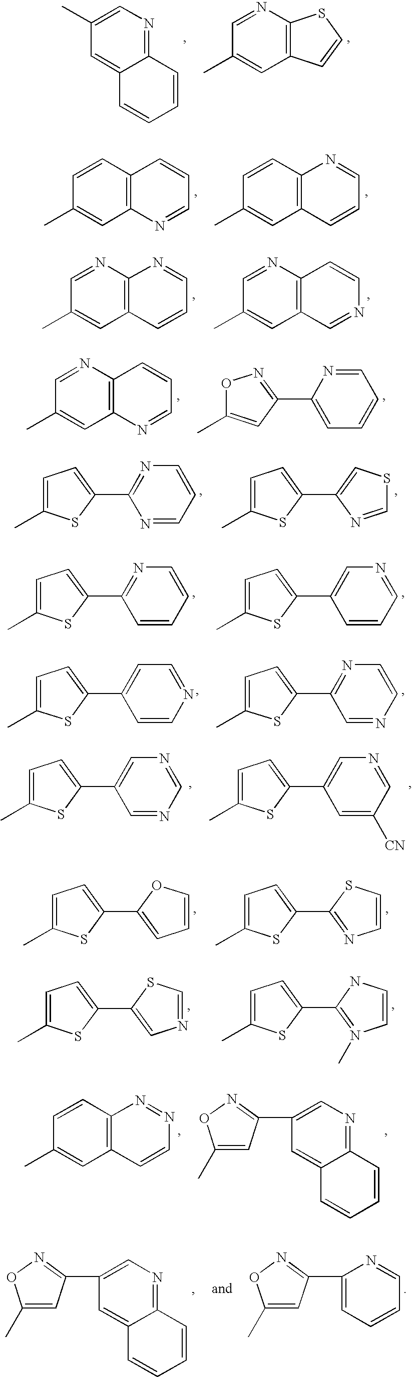

8. The method of claim 1 wherein the macrolide or ketolide has a structural formula 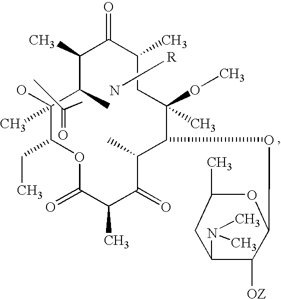
wherein R is 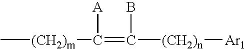
m and n are individually integers from 0 to 6, A and B are individually a member selected from the group consisting of hydrogen, halogen, and alkyl of 1 to 8 carbon atoms, the double bond geometry being E or Z or E+Z or A and B for a third bond between the carbon atoms to which they are attached, Ar is selected from the group consisting of a) carbocyclic aryl or up to 18 carbon atoms optionally substituted with at least one member of the group consisting of free carboxy, alkoxycarbonyl, carboxy salified with a nontoxic, pharmaceutically acceptable base, amidified carboxy, —OH, halogen, —NO₂, —CN, alkyl, alkenyl, alkynyl, alkoxy, alkenyloxy, alkynyloxy, alkylthio, alkenylthio, and alkynylthio of up to 12 carbon atoms, N-alkyl, N-alkenyl, and N-alkynyl of up to 12 carbon atoms and cycloalkyl of 3 to 12 carbon atoms, all optionally substituted with at least one halogen and 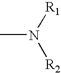
R₁ and R₂ are individually selected from the group consisting of hydrogen, alkyl of 1 to 12 carbon atoms, carbocyclic aryl, aryloxy, arylthio, heterocyclic aryl, and aryloxy and arylthio containing at least one heteroatom, all optionally substituted as above and b) heterocyclic aryl having at least one heteroatom optionally substituted with at least one of the above substituents, Z is hydrogen or acyl or an organic carboxylic acid of 1 to 18 carbon atoms and their nontoxic, pharmaceutically acceptable acid addition salts.

9. The method of claim 1 wherein the macrolide or ketolide has a structural formula 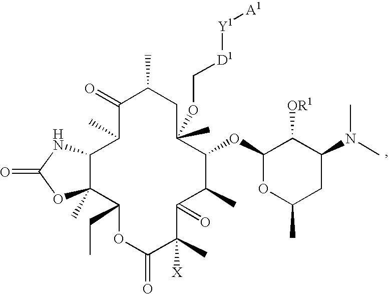
or a therapeutically acceptable salt or prodrug thereof, wherein
X is selected from hydrogen and fluoride;
D¹ is selected from CH═CH or C≡C;
y¹ is selected from isoxazole, oxazole, isothiazole, dihydroisoxazole, and dihydrooxazole;
A¹ is selected from aryl and heteroaryl; and
R¹ is selected from hydrogen and R^{p}, wherein R^{p} is a hydroxyl protecting group.

10. The method of claim 1 wherein the macrolide or ketolide has a structural formula 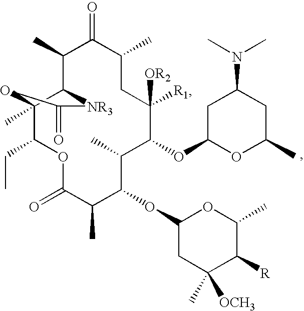
wherein R and R₁ are —OH or —O-acyl of an organic carboxylic acid:of 2 to 20 carbon atoms, R₂ is hydrogen or methyl, R₃ is —(CH₂)ₘ—R₄ or 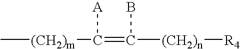
or —N—(CH₂)_{q}—R₄, m is an integer from 1 to 6, a, p, and q are individually an integer from 0 to 6, A and B are individually selected from the group consisting of hydrogen, halogen, and alkyl of 1 to 8 carbon atoms with the geometry of the double bond being E or Z or a mixture of E and Z or A and B form a triple bond, R₄ is an optionally substituted mono- or polycyclic heterocycle and their nontoxic, pharmaceutically acceptable acid addition salts.

11. The method of claim 1 wherein the macrolide or ketolide has a structural formula 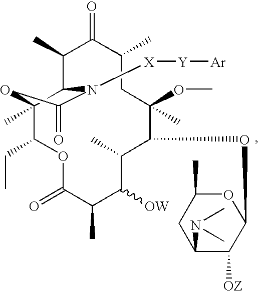
wherein X represents a CH₂ or SO₂ radical or an oxygen atom, Y represents a (CH₂)ₘ(CH═CH)ₙ(CH₂)ₒ radical, with m+n+o≦8, n=0 or 1,
Ar represents an optionally substituted aryl radical, and
W represents a hydrogen atom, or the remainder of a carbamate function 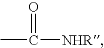
wherein R″ represents an alkyl radical containing up to 8 carbon atoms or an optionally substituted aryl radical,
Z represents a hydrogen atom or the remainder of an acid, as well as their addition salts with acids.

12. The method of claim 1 wherein the macrolide or ketolide has a structural formula 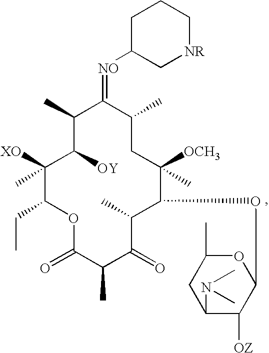
wherein X and Y are hydrogen or together form 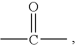
R is 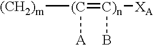
m is an integer from 0 to 20, n is 0, 1, 2, or 3, A and B are individually selected from the group consisting of hydrogen, halogen, alkyl of 1 to 8 carbon atoms and aryl of 6 to 8 carbon atoms with the double bond geometry being E or Z or a mixture of E and Z or A and B form a third bond between the carbons to which they are attached, X_{A} is selected from the group consisting of alkyl, alkenyl, and alkynyl of 6 to 20 carbon atoms optionally interrupted with at least one heteroatom and optionally substituted with at least one halogen, cycloalkyl of 3 to 8 carbon atoms optionally substituted by a carbocyclic aryl, halogen, —CN, —OR₃, —COR₄, —COOR₅, —SR₆, SOR₇, —SO₂R₈, 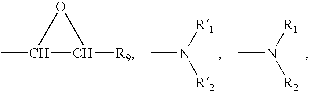
—OC(Ar)₃, and a carbocyclic aryl and heterocyclic aryl optionally substituted, R₃, R₄, R₅, R₆, R₇, R₈, and R₉ are individually selected from the group consisting of hydrogen, alkyl of 1 to 8 carbon atoms optionally interrupted by at least one heteroatom and optionally substituted by at least one halogen, carbocyclic, and heterocyclic aryl and aralkyl of up to 14 carbon atoms optionally substituted with at least one member of the group consisting of free, salified, esterified, or amidified carboxy, —OH, halogen, —NO₂, —CN, alkyl, alkenyl, alkynyl, alkoxy, alkenyloxy, alkynyloxy, alkylthio, alkenylthio, alkynylthio, —SO-alkyl, —SO-alkenyl, —SO-alkynyl, —SO₂-alkyl, —SO₂alkenyl, and —S₂-alkynyl of up to 12 carbon atoms, all optionally substituted with at least one halogen, carbocyclic, and heterocyclic, aryl, O-aryl, and —S-aryl of up to 14 carbon atoms, R′₁ and R′₂ are individually selected from the group consisting of hydrogen and alkyl of 1 to 12 carbon atoms, R₁ and R₂ are individually selected from the group consisting of hydrogen, alkyl of 1 to 20 carbon atoms, —COAlk, and —COO-Alk, aryl, —CO-aryl, —COO-aryl, aralkyl, —CO-aralkyl, and —COO-aralkyl of up to 14 carbon atoms, Alk of alkyl of 1 to 8 carbon atoms, or R₁ and R₂ together with the nitrogen to which they are attached form ring of 3 to 8 members optionally containing a second heteroatom and optionally substituted with the above aryl substituents, Ar is a carbocyclic aryl optionally substituted with at least one of the above aryl substituents, Z is hydrogen or aryl of an organic carboxylic acid of up to 18 carbon atoms and their nontoxic, pharmaceutically acid addition salts.

13. The method of claim 1 when the macrolide or ketolide comprises a macrolide.

14. The method of claim 1 wherein the tuberculoses comprises latent tuberculosis, active tuberculosis, or multidrug-resistant tuberculosis.

15. The method of claim 1 further comprising administering a therapeutically effective amount of a second drug useful in treatment of tuberculosis.

16. The method of claim 15 wherein the second drug is selected from the group consisting of isoniazid, rifampin, pyrazinamide, streptomycin, and mixtures thereof.

17. The method of claim 15 when the macrolide or ketolide and the second drug are administered simultaneously.

18. The method of claim 15 when the macrolide or ketolide and the second drug are administered sequentially.

19. An article of manufacture comprising:
(a) a packaged composition comprising a macrolide, ketolide, or mixture thereof;
(b) an insert providing instructions for administration of the packaged composition of (a) to treat tuberculosis; and
(c) a container for (a) and (b).

20. An article of manufacture comprising:
(a) a packaged composition comprising a macrolide, ketolide, or mixture thereof;
(b) a packaged composition comprising a second therapeutic agent useful in a treatment of tuberculosis;
(c) an insert providing instructions for a simultaneous or sequential administration of (a) and (b) to treat tuberculosis; and
(d) a container for (a), (b), and (c).

21. A compound selected from the group consisting of a compound disclosed in Table 1 of the specification and a compound disclosed in Table 3 of the specification.
